(19) United States
(12) Patent Application Publication
(10) Pub. No.: US 2010/0062946 A1
(43) Pub. Date: Mar. 11, 2010

(54) GENOME-WIDE METHOD FOR MAPPING OF ENGAGED RNA POLYMERASES QUANTITATIVELY AND AT HIGH RESOLUTION

(76) Inventors: John T. Lis, Ithaca, NY (US); Leighton J. Core, Freeville, NY (US)

Correspondence Address: MARJAMA MULDOON BLASIAK & SULLIVAN LLP, 250 SOUTH CLINTON STREET, SUITE 300, SYRACUSE, NY, 13202, US

(21) Appl. No.: 12/554,472

(22) Filed: Sep. 4, 2009

Related U.S. Application Data
(60) Provisional application No. 61/095,070, filed on Sep. 8, 2008.

(51) Int. Cl. C12Q 1/68; C40B 30/00

(52) U.S. Cl. 506/7; 435/6

(57) ABSTRACT

A method is provided for detecting genome-wide transcriptionally-engaged RNA polymerases. The method can also be used to assess status and regulation of gene promoters. The method comprises permeabilizing a cell of interest or isolating the nucleus from a cell of interest; performing a nuclear run-on (NRO) reaction with the permeabilized cell or isolated nucleus, wherein a purifiable nucleotide analog is added to the NRO reaction; optimizing the number of bases traveled by engaged polymerases for high resolution and low bias for nucleotide content of transcribed sequences by limiting a second nucleotide concentration or duration of the NRO reaction; isolating NRO-RNA from the NRO reaction; hydrolyzing the NRO-RNA isolated from the NRO reaction to optimize resolution of polymerase location; selecting hydrolyzed NRO-RNA with a solid support to obtain an enriched, purified fraction of the hydrolyzed NRO-RNA; enzymatically repairing the hydrolyzed NRO-RNA; and ligating the hydrolyzed NRO-RNA to compatible adapter oligos.

### CROSS-REFERENCE TO RELATED APPLICATIONS

This application claims priority to and the benefit of co-pending U.S. provisional patent application Ser. No. 61/095,070, entitled Genome-Wide. Method for Mapping of Engaged RNA Polymerases Quantitatively and at High Resolution, filed Sep. 8, 2008, which is incorporated herein by reference in its entirety.

### STATEMENT REGARDING FEDERALLY SPONSORED RESEARCH OR DEVELOPMENT

The disclosed invention was made with government support under contract no. GMO25232 from the National Institutes of Health. The government has rights in this invention.

### 1. TECHNICAL FIELD

The present invention relates to methods for measuring the production of nascent RNA. The invention also relates to methods for assessing the status of gene promoters and their mode of regulation. The invention further relates to methods for mapping positions of RNA polymerases.

### 2. BACKGROUND OF THE INVENTION

Elucidation of the genome wide location and abundance of transcription of coding and non-coding RNAs by RNA polymerases is a rapidly growing field of research. The advent of whole-genome microarrays and ultra high-throughput sequencing technologies provide remarkable advances in the efficiency of mapping the distribution of transcription factors, nucleosomes and their modifications, and RNA transcripts throughout genomes. Whole genome mapping of accumulated RNA transcripts by microarray hybridization or high throughput sequencing methods are beginning to show that the genome is highly transcribed compared to previous estimates, with some notable features being novel transcription units and unannotated sense/antisense transcript pairs. These recent discoveries indicate that the origin and function of transcribed RNAs is still being defined, thus independent methods that can comprehensively document sites of transcription are of utmost importance for understanding genome function and regulation during both homeostasis and animal development.

Several studies using the chromatin immunoprecipitation (ChIP) assay coupled to genomic DNA microarrays (ChIP-chip) have shown that RNA Polymerase II (Pol II) is present at disproportionately higher levels near the 5′ end of many genes relative to downstream portions. This technique locates Pol II complexes but, cannot necessarily determine whether they are engaged in transcription or not. Small-scale analyses using independent methods, such as nuclear run-ons or potassium permanganate footprinting, have shown that this distribution likely represents a transcriptionally engaged but paused Pol II. This promoter-proximal pausing is a mechanism through which transcription of genes can be regulated at the stage of elongation rather than recruitment of Pol II. However, no assay exists to test this hypothesis on the genomic scale.

Nuclear run-on (NRO) assays are traditionally used to measure transcribing polymerases over specific targeted regions of the genome. Traditionally, nuclei are isolated, endogenous nucleotides are removed by washing, and radionucleotides are added back for short times allowing transcriptionally engaged polymerases to resume elongation. Thus all new transcription is produced by polymerases that are engaged at the time of nuclear isolation. The RNA is then isolated and hybridized to filters containing genes or gene regions of interest. The NRO represents the level of transcriptionally-engaged Pol II at the time of nuclei isolation, thereby defining the level of expression of certain genes. However a NRO cannot work in a genome-wide manner.

Previous attempts at scale-up of NRO assays have entailed hybridizing radiolabeled NRO RNAs to cDNA probes spotted on macroarrays (NRO-cDNA hybridizations) to analyze how steady state transcription of genes relates to mRNA accumulation. These methods can give reasonable approximations for steady state transcription levels. However, they suffer from low sensitivity, lack of whole genome coverage, and zero resolution within gene regions. Whole genome coverage is important for detection of novel transcription units as well as transcripts that are not present in cDNA libraries. The lack of resolution of cDNA arrays is of concern since genes that have a promoter proximal paused Pol II and are not producing full-length transcripts will produce detectable signal on these arrays that does not reflect actual levels of full-length transcription of those genes.

### 3. SUMMARY OF THE INVENTION

A method for performing a genome-wide nuclear run-on assay in a cell of interest is provided. In one embodiment, the method can comprise:

1) permeabilizing the cell of interest or isolating the nucleus from the cell of interest;
2) performing a nuclear run-on (NRO) reaction with the permeabilized cell or the isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration or duration of the NRO reaction;
4) isolating NRO-RNA from the NRO reaction;
5) hydrolyzing the NRO-RNA isolated from the NRO reaction to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA; and
8) ligating the hydrolyzed NRO-RNA to compatible adapter oligos.

In another embodiment, the cell of interest is a plurality of cells of interest and the step of permeabilizing comprises permeabilizing the plurality.

In another embodiment, the cell of interest is a plurality of cells of interest and the step of isolating the nucleus comprises isolating nuclei from the plurality.

In another embodiment, the step of isolating the nucleus comprises chemical or mechanical disruption of the outer cell membrane.

In another embodiment, the solid support is a bead support, column matrix, membrane support, biochip, microtiter plate or microfluidic device.

In another embodiment, the purifiable nucleotide analog comprises a purifiable affinity tag.

In another embodiment, the purifiable nucleotide analog is 5-Bromo-UTP (BrU) and the second nucleotide is not U or an analog thereof.

In another embodiment, the step of isolating the NRO-RNA comprises using a moiety that binds BrU contained within the NRO-RNA.

In another embodiment, the moiety is an antibody, an aptamer or a protein that reversibly binds BrU contained within the NRO-RNA.

In another embodiment, the step of enzymatically repairing the hydrolyzed NRO-RNA comprises removing the 5′ cap.

In another embodiment, removing the 5′ cap is accomplished through tobacco acid pyrophosphatase (TAP) treatment.

In another embodiment, the step of enzymatically repairing the hydrolyzed NRO-RNA comprises adding a 5′-phosphate (5′-P).

In another embodiment, adding the 5′-P is accomplished through neutral pH T4 polynucleotide kinase (T4 PNK) treatment.

In another embodiment, the step of enzymatically repairing the hydrolyzed NRO-RNA comprises removing a 3′-phosphate (3′-P).

In another embodiment, removing the 3′-P is accomplished through low pH T4 PNK treatment.

In another embodiment, the method comprises reverse transcribing the NRO-RNA ligated to the compatible adapter oligos.

In another embodiment, the method comprises producing a NRO-cDNA second strand by DNA extension.

In another embodiment, the method comprises amplifying the double-stranded NRO-cDNA thereby producing a NRO-library.

In another embodiment, the method comprises sequencing the amplified NRO-library.

In another embodiment, the method comprises mapping one or more sequence reads to a reference genome.

In another embodiment, the method comprises determining position, orientation or number of hits for the sequence read.

In another embodiment, the hydrolyzing step comprises base hydrolyzing.

In another embodiment, the hydrolyzing step comprises RNase hydrolyzing.

In another embodiment, the step of selecting hydrolyzed NRO-RNA comprises triple-selecting the hydrolyzed NRO-RNA.

In another embodiment, the method comprises analyzing the hydrolyzed NRO-RNA ligated to compatible adapter oligos using sequencing analysis or microarray analysis.

In another embodiment, the sequencing analysis is massively parallel sequencing analysis.

In another embodiment, the analysis is microarray analysis and the NRO-RNA is ligated to an oligo containing a promoter for an RNA polymerase.

In another embodiment, the method comprises analyzing production of nascent RNA.

In another embodiment, the method comprises determining transcriptionally-engaged polymerase density.

In another embodiment, the production of nascent RNA is compared to accumulated mRNA levels to identify genes regulated by mRNA turnover.

In another embodiment, the method comprises, after the step of amplifying the NRO-RNA, the step of purifying the amplified NRO-RNA by PAGE purification.

In another embodiment, the method comprises treating the isolated nucleus with RNase prior to the step of running the NRO reaction; and identifying polymerase active sites after the step of ligating the hydrolyzed NRO-RNA to compatible adapter oligos.

In another embodiment, the purifiable nucleotide analog does not allow further elongation.

A method for identifying a transcription start site in the genome of a cell of interest is also provided. The method can comprise the steps of:

1) permeabilizing the cell of interest or isolating the nucleus from the cell of interest;
2) performing a nuclear run-on (NRO) reaction with the permeabilized cell or the isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration or duration of the NRO reaction;
4) isolating NRO-RNA from the NRO reaction;
5) hydrolyzing the NRO-RNA isolated from the NRO reaction to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA;
8) selecting capped NRO-RNAs through enzymatic enrichment by the oligo-capping method; and
9) ligating the hydrolyzed NRO-RNA to compatible adapter oligos.

A method for identifying the position of an active site of an engaged RNA polymerase in the genome of a cell of interest is also provided. The method can comprise the steps of:

1) permeabilizing the cell of interest or isolating the nucleus from the cell of interest;
2) hydrolyzing RNA in the permeabilized cell or the isolated nucleus with an RNase;
3) performing a nuclear run-on (NRO) reaction with the permeabilized cell or the isolated nucleus, wherein a purifiable nucleotide analog is added to the NRO reaction;
4) optimizing the number of bases traveled by engaged polymerases for high resolution and low bias for nucleotide content of transcribed sequences by limiting a second nucleotide concentration or duration of the NRO reaction;
5) isolating NRO-RNA from the NRO reaction;
6) selecting hydrolyzed NRO-RNA with a solid support to obtain a highly enriched and purified fraction of the hydrolyzed NRO-RNA;
7) enzymatically repairing the hydrolyzed NRO-RNA by removing a 5′ cap from the NRO-RNA and adding a 5′-P to the NRO-RNA; and
8) ligating the hydrolyzed NRO-RNA to compatible adapter oligos.

In one embodiment, the step of enzymatically repairing the hydrolyzed NRO-RNA by removing the 5′ cap from the NRO-RNA and adding the 5′-P to the NRO-RNA comprises TAP treatment and neutral pH PNK treatment.

A method for mapping a site of co-transcriptional cleavage that delineates the 3′ end of an mRNA is also provided. The method can comprise the steps of:

1) permeabilizing the cell of interest or isolating the nucleus from the cell of interest;
2) performing a nuclear run-on (NRO) reaction with the permeabilized cell or isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration or duration of the NRO reaction;
4) isolating NRO-RNA from the NRO reaction;
5) optionally hydrolyzing the NRO-RNA isolated from the NRO reaction to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA removing a 3′-P from the hydrolyzed NRO-RNA; and
8) ligating the hydrolyzed NRO-RNA to compatible adapter oligos.

In one embodiment, the method comprises, after the step of ligating the hydrolyzed NRO-RNA to compatible adapter oligos, the step of amplifying the NRO-RNA.

In another embodiment, the method comprises the step of performing reverse transcription after the step of ligating the hydrolyzed NRO-RNA to compatible adapter oligos, wherein the ligating step comprises addition of a RNA oligomer to the 5′-end of the NRO-RNA and addition of an RNA oligomer to the 3′-end of the NRO-RNA.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated or enlarged to facilitate an understanding of the invention.

FIG. 1. Schematic of Global Run-on (GRO) combined with sequencing technology (GRO-seq).

FIG. 2. Incorporation of Br-UTP in a nuclear run-on.

FIG. 3. Control of nuclear run-on distance by limiting nucleotide concentration.

FIG. 4. Bead binding efficiency in response to [CTP] titration.

FIG. 5. Binding and elution of base-hydrolyzed BrU-RNA to α-BrdU beads.

FIG. 6. Denaturing PAGE analysis of fractions from GRO-seq library preparation.

FIG. 7. Example of amplified NRO-library cDNA prior to PAGE purification.

FIG. 8. Example of GRO-seq data as viewed in the UCSC genome browser.

FIG. 9. Example of the specificity of bead isolation of BrU-NRO-RNA.

FIG. 10. Comparison of GRO-seq read density in exons versus introns.

FIG. 11. Correlation of GRO-seq biological replicates

FIG. 12. Plot of interlibrary correlation versus read trimming.

FIG. 13. Background calculation by low-density windows.

FIG. 14. Summary of the fraction of GRO-seq reads mapping in or near gene regions.

FIG. 15. Identification of three types of antisense transcription by analysis of data generated by GRO-seq.

FIG. 16. Alignment of GRO-seq hits relative to TSSs

FIG. 17. Alignment of GRO-seq hits to annotated 3′-ends.

FIG. 18. GRO-seq activity versus expression microarray scatter plots.

FIG. 19. RT-qPCR validation of GRO-seq levels.

FIG. 20. Classification of genes based on total activity and polymerase distribution.

FIG. 21. Correlation of promoter-proximal transcription patterns with gene activity.

FIG. 22. Fraction of paused genes and active genes by gene activity decile.

FIG. 23. Gene ontology of paused genes.

FIGS. 24A-D. GRO-seq profiles for known paused genes.

FIG. 25. Distribution of GRO-seq relative to Pol II ChIP data and histone modification data.

FIG. 26. Example of a novel promoter identified by ChIP and GRO-seq.

FIG. 27. Schematic of method to map polymerases with near nucleotide resolution.

FIG. 28. Table 1. Background calculation by tabulating GRO-seq reads in gene deserts.

FIG. 29. Table 2. Summary of GRO-seq and microarray gene activity calls.

FIG. 30. Table 3. Pairwise correlations between Gene Activity, Pausing, Divergent transcription, and CpG island promoters.

### 5. DETAILED DESCRIPTION OF THE INVENTION

A method is provided for mapping the position, direction and abundance of engaged RNA polymerases in a cell of interest under any condition and provides a snapshot of steady-state transcription level. The method, referred to herein as the Global Run-On (GRO) method, can be used to detect changes in expression at a resolution that is unattainable by hybridization and sequencing methods and also provides resolution within genes and whole genome coverage that is not attainable with NRO-cDNA hybridizations. Unlike ChIP experiments, the GRO method can be used to identify genes that are regulated through promoter pausing, and identify novel sites of transcription throughout the genome.

Any eukaryotic cell or organelle thereof containing a transcribed genome of interest can be analyzed by the GRO method, e.g., plant cells, animal cells, chloroplasts, mitochondria, etc.

The method can be used, in one embodiment, to analyze all transcripts following a nuclear run-on (NRO) assay. Traditional NRO assays, by contrast, analyze only select transcripts.

Using the GRO method, transcriptionally-engaged polymerase density throughout the genome can be analyzed by tracking the associated nascent RNA. The GRO method can be used to map the position, direction and abundance of transcriptionally engaged RNA polymerases in a genome-wide manner. This provides a quantitative and highly sensitive snapshot of gene expression at the level of transcription and also allows both the detection of rare or unstable transcripts that are not easily detected in accumulated RNA pools. The method can be used to track steady-state production of nascent RNA. The data obtained can be compared to accumulated mRNA levels to examine the extent with which particular genes are regulated by mRNA turnover.

Unlike NRA assays which can only detect expression level of one or several desired genes, the GRO method enables the investigator to perform a genome-wide nuclear run-on assay in any cell, under any condition, in quantitative and sensitive manner.

The data obtained from GRO analyses can be used to identify genes regulated by accelerated or low rates of RNA turnover or to identify genes that are regulated through promoter-proximal pausing, thereby identifying novel sites of transcription currently undetectable by methods currently available in the art.

5.1 Method for Analyzing Transcriptionally Engaged Polymerases

A method is provided for detecting transcriptionally-engaged RNA polymerases. The method, referred to herein as the Global Run-On (GRO) method, improves the traditional nuclear run-on assay (NRO) and is designed to document transcriptionally-engaged RNA polymerases in a genome-wide, quantitative, and highly-sensitive manner. In addition, the method can be used to assess the status of gene promoters and their mode of regulation.

Through previous studies using the chromatin immunoprecipitation (ChIP) assay coupled to genomic DNA microarrays (ChIP-chip), RNA Polymerase II (Pol II) is known to be present at disproportionately higher levels near the 5′ end of many genes relative to downstream portions. The ChIP-chip technique locates Pol II complexes but cannot necessarily determine whether they are engaged in transcription or not. Small-scale analyses using independent methods, such as conventional nuclear run-ons or potassium permanganate footprinting, have shown that this distribution likely represents a transcriptionally engaged but paused Pol II. Through the mechanisms of promoter-proximal pausing, transcription of genes can be regulated at the stage of elongation rather than at the stage of recruitment of Pol II. The GRO method can be used to evaluate this promoter-proximal pausing mechanism for all genes in a single experiment.

The GRO method is a genome-wide version of a NRO assay. NRO assays are traditionally used to measure the density of transcribing polymerases over specific targeted regions of the genome, and variations of the assay are capable of mapping the position of polymerases with high precision. Traditionally, nuclei are isolated, endogenous nucleotides are removed by washing, and radionucleotides are added back for short times allowing transcriptionally engaged polymerases to resume elongation. The anionic detergent sarkosyl, which does not interfere with elongating polymerases, is often added to the nuclear run-on reaction to ensure that new transcription initiation events do not occur, and to remove physical impediments that can block elongation. Thus all new transcription is produced by polymerases that are engaged at the time of nuclear isolation. The RNA is then isolated and hybridized to filters containing genes or gene regions of interest. These measurements have been shown to represent the level of transcriptionally-engaged Pol II at the time of nuclei isolation, thereby defining the level of expression of genes. Additionally, these measurements identify Pol II that is paused at the 5′ ends of genes, as well as the distance Pol II travels beyond the 3′-ends of genes prior to termination. The GRO method, unlike methods known in the art, documents these characteristics of transcription in a genome-wide manner.

In addition, the distribution of transcribing polymerases within genes provides information on how a particular gene is regulated, and when combined with knowledge of promoter DNA sequences, transcription factor binding sites, and nucleosomes and their modifications, can further knowledge of how these elements cooperate to specify distinct transcriptional outcomes.

The GRO method can be used to map the position, direction and abundance of transcriptionally engaged RNA polymerases in any cell under any condition. The GRO method provides a snapshot of steady-state transcription level and therefore can be used to follow changes in expression at a high temporal resolution that is unattainable by hybridization and sequencing methods that analyze accumulated RNA. Data obtained by using the GRO method also provides resolution within genes, sensitivity, and whole genome coverage that is unattainable with NRO-cDNA hybridizations. The data obtained from GRO, unlike the data obtained chromatin immunoprecipitation (ChIP), can be used to unambiguously identify genes that are regulated through promoter-proximal pausing and to identify novel sites of transcription throughout the genome with sensitivity that is presently unattainable by any other method.

In one embodiment, the GRO method comprises:

1) permeabilizing the cell of interest or isolating the nucleus from the cell of interest;
2) performing a nuclear run-on (NRO) reaction with the permeabilized cell or the isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration (e.g., C, U, A or G) or duration of the NRO reaction;
4) isolating NRO-RNA from the NRO reaction;
5) hydrolyzing the NRO-RNA isolated from the NRO reaction to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA; and
8) ligating the hydrolyzed NRO-RNA to compatible adapter oligos.

In another embodiment, the GRO method can be used to detect transcription start sites.

In another embodiment, the GRO method can be used to map the active site of an engaged RNA polymerase at near-nucleotide resolution.

In another embodiment, the GRO method can be used map sites of co-transcriptional cleavage that delineate the 3′ end of mRNAs.

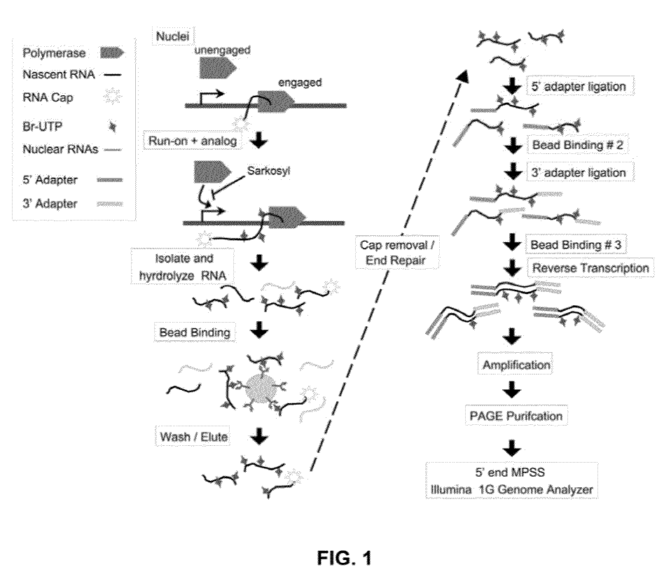

The GRO method provides a genome-wide view of transcriptionally engaged polymerases. FIG. 1 is a schematic of one embodiment of the GRO method. In this embodiment, GRO is combined with massively parallel sequencing (‘GRO-seq’) on an Illumina 1G genome analyzer.

### Step 1: Permeabilizing cells or isolating nuclei from cells

The GRO method comprises the step of either permeabilizing a cell (or a plurality of cells) of interest or isolating a nucleus from a cell (or from a plurality of cells) of interest. Methods for permeabilizing the cell outer membrane are well known in the art.

Isolation of the nucleus can be performed by chemical and/or mechanical disruption of the outer cell membrane to release the nuclei from the cell. Such disruption is well known in the art (see, e.g., U.S. Pat. No. 4,906,561, Mar. 6, 1990 to Thornthwaite; U.S. Pat. No. 4,668,618, May 26, 1987 to Thornthwaite; U.S. Pat. No. 7,374,881, May 20, 2008 to Mitsuhashi; U.S. Pat. No. 5,972,613, Oct. 26, 1999 to Somack et al.; U.S. Pat. No. 5,128,247, Jul. 7, 1992 to Koller; U.S. Pat. No. 6,413,720, Jul. 2, 2002 to Pardinas et al.).

Nuclei can then be enriched from cellular debris through differential centrifugation, which causes the nuclei to settle to the bottom of a tube faster than the debris.

Chemical disruption of the cell membrane can achieved through methods known in the art, such as treatment with mild detergents that disrupts the cell membrane.

Mechanical disruption of the cell membrane can also be achieved through methods known in the art. For example, the cells can be further disrupted by douncing, which involves forcing the cells through a tight space (0.1-0.15 mm) in a glass homogenizer with a pestle. The mechanical force applied causes the cell membranes to break apart, releasing the nuclei and other cellular components.

The nuclei are then washed several times to remove endogenous nucleotides, which is important for stopping elongation of polymerases as well as for subsequent steps of the GRO method.

Step 2: Performing a Nuclear-Run-on (NRO) Reaction

The GRO method comprises the step of performing a nuclear-run-on (NRO) reaction with the permeabilized cell or the isolated nucleus (or a plurality of permeabilized cells or isolated nuclei), wherein a purifiable nucleotide analog is added to the NRO reaction. Unlike NRO reactions, in which radionucleotides cannot be used in high density microarrays, the GRO method can employ a nucleotide analog with a purifiable affinity tag that is added during the NRO reaction step and that can be used in high density microarray platforms.

The actual amount of new RNA produced during a NRO reaction represents less than 1% of the RNA in the nucleus. Thus NRO-RNA must be highly purified in order to minimize background signal. Traditional nuclear run-on reactions use radionucleotides that allow the direct visualization of NRO-RNA when hybridized to macroarray filters. However, radionucleotides cannot be used in high density microarray platforms, and do not allow specific isolation of NRO-RNAs away from contaminate RNAs.

To solve this problem, a nucleotide analog with a purifiable affinity tag can be added during the NRO reaction step of the method. The NRO-RNA can then be specifically isolated by standard affinity chromatography and analyzed by a variety of methods.

In one embodiment, the purifiable nucleotide analog can be conjugated to another moiety known in the art that is purifiable (e.g., the hormone digoxigenin). According to this embodiment, an antibody to the conjugated moiety (e.g., digoxigenin) can be used for selection.

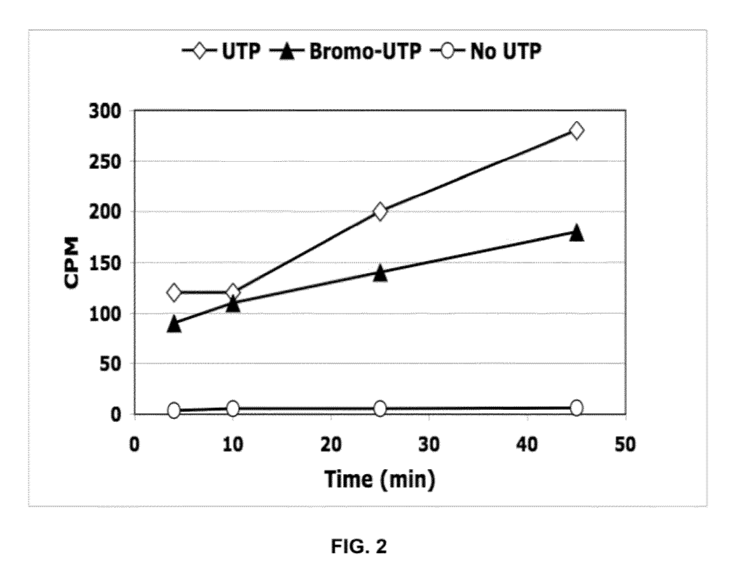

In one embodiment, the nucleotide analog 5-Bromo-UTP (BrU) is used in place of UTP during the NRO reaction step, which can be used by RNA polymerases as a substrate (FIG. 2).

The incorporation of BrUTP into newly synthesized mRNA during transcription with high affinity is known in the art (see, e.g., U.S. Pat. No. 5,660,985, Aug. 26, 1997 to Pieken et al.; U.S. Pat. No. 6,124,099, Sep. 26, 2000 to Heckman et al.; U.S. Pat. No. 6,958,217, Oct. 25, 2005 to Pedersen).

FIG. 2 shows incorporation of Br-UTP in a nuclear run-on. Polymerases were run-on in nuclei supplemented with Sarkosyl, ATP, GTP, α-³²P-CTP and UTP (open diamonds), Br-UTP (closed triangles), or no UTP (open circles). Separate reactions were setup for each time point and the reactions were stopped at 5, 10, 25 or 45 min. The RNAs were isolated, and the radioactivity incorporated was assayed by scintillation counting.

Step 3: Controlling the Distance Polymerases Travel by Limiting a Second Nucleotide Concentration and Duration of the Reaction

The GRO method comprises the step of optimizing the number of bases traveled by engaged polymerases for high resolution and low bias for nucleotide content of transcribed sequences by limiting a second nucleotide concentration (e.g., C, A, U or G or duration of the NRO reaction.

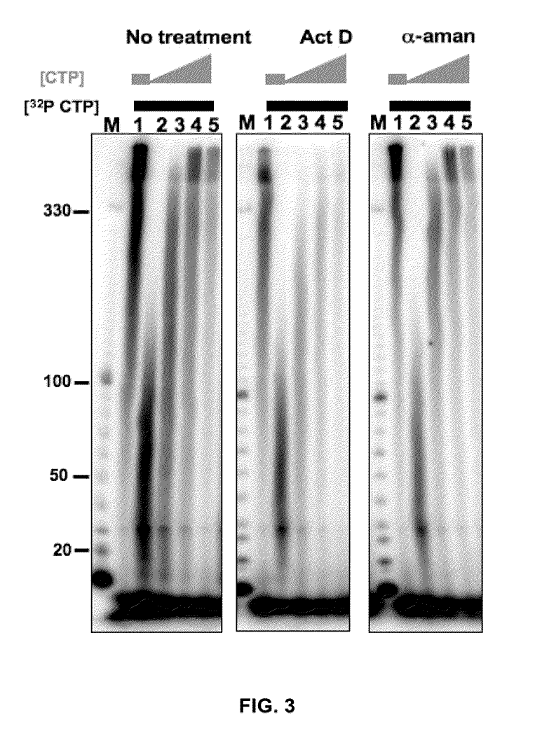

To obtain high resolution of polymerase positions at the time of nuclei isolation, the distance the polymerase elongates during the reaction is preferably kept as short as possible. This distance can be controlled by limiting the concentration of one or all the nucleotides in the reaction (FIG. 3), and by altering the time the reaction is allowed to proceed. Methods for calculating preferable concentrations and reaction times are well known in the art.

FIG. 3 shows control of nuclear run-on distance by limiting nucleotide concentration. Nuclei were pre-treated with RNase to reduce the nascent RNA to ˜20 nucleotides, washed, and then allowed to run-on in separate reactions containing a α-32P-CTP and cold CTP for a total of 0.65 μM (Lane 2), 1 μM (lane 3), 5 μM (lane 4) or 25 μM (lane 5). Non-RNase treated nuclei supplemented with 1 μM total CTP were used as a control (Lane 1). Cells were treated with Act-D and nuclei were treated with α-amanitin. Actinomycin D primarily inhibits Pol I in these conditions, and α-amanitin inhibits Pol II. Thus the distance travel by each of the three main RNA polymerases (I, II, and III), can be deduced by this experiment.

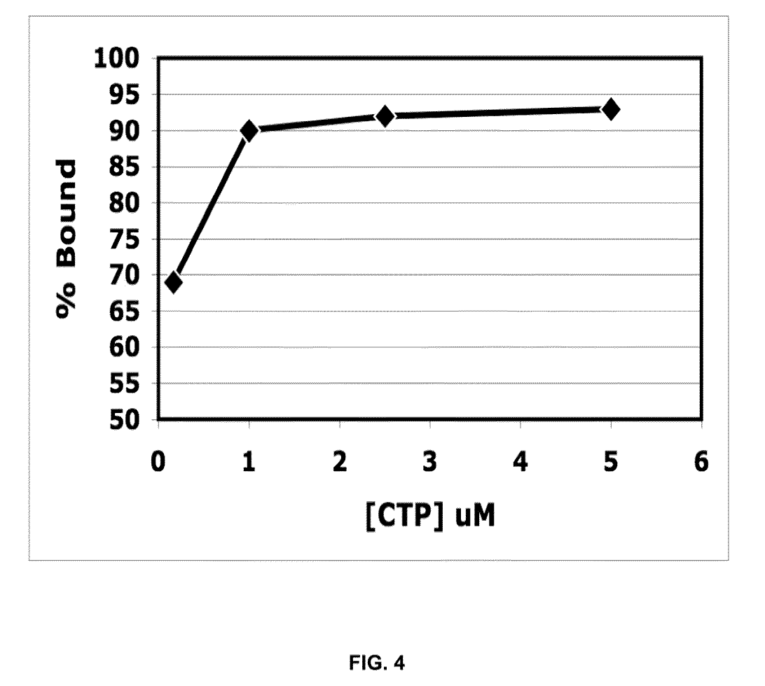

If only one purifiable nucleotide analog is to be used, the distance the polymerases travel should be optimized to prevent bias in the nucleotide composition of the isolated NRO-RNAs (FIG. 4). Thus, the preferable distance polymerases are allowed to elongate is preferably determined as the shortest distance that allows the highest efficiency of isolating all the NRO-RNA produced during the reaction. This distance was found to be ˜100 bases in length.

FIG. 4 shows bead binding efficiency in response to [CTP] titration. NRO reactions were performed as described in FIG. 3, but without pre-treatment with RNase. Run-on RNAs from each sample were base-hydrolyzed and bound to equivalent amounts of beads. The bound and unbound fractions were monitored for radioactivity by scintillation counting. The percent bound (y-axis) was calculated relative to input fractions and is displayed relative to the concentration of CTP in the reaction (x-axis).

Step 4: Isolating NRO-RNA from the NRO Reaction

The GRO method comprises isolating NRO-RNA from the NRO reaction. Isolation of NRO-RNA is well known in the art and exemplary methods are described in Section 6.

Step 5: Hydrolyzing the NRO-RNA Isolated from the NRO Reaction to Optimize Resolution of Polymerase Location

The GRO method comprises hydrolyzing the NRO-RNA isolated from the NRO reaction to optimize resolution of polymerase location. Any hydrolysis method known in the art can be used. In specific embodiments, base hydrolysis or RNase hydrolysis is used. Base hydrolysis can also be substituted with sonication or addition of divalent cations and heat.

In one embodiment, isolated RNA from a nuclear run-on containing Br-UTP and α-³²P-CTP can be hydrolyzed to an average size of 100 bases.

Elongating RNA polymerases are associated with nascent RNAs that are on average, several kilobases (kb) in length. To obtain a high resolution snapshot of polymerase locations in the genome, the GRO method utilizes base hydrolysis of the NRO-RNA. Base hydrolysis to a size that is equal to the distance the polymerases elongate during the NRO reaction (in this case ˜100 bases) results in small RNA fragments that represent the original location of the polymerase within 30 bases.

As described above, both base hydrolysis and RNase hydrolysis can be used to hydrolyze RNA. In certain embodiments, an RNase enzyme can also be used to hydrolyze. RNA in the nucleus prior to Step 2, performing the NRO reaction. The polymerase protects a small portion of the RNA (15-20 bases), then the RNase enzyme can be washed away and the NRO reaction can be run. This can be done, for example, when the GRO method is used to determine the distance the polymerase travels, and when mapping the polymerase active site based on the digestion pattern. If base hydrolysis was performed instead. RNase in these embodiments, the polymerase would be destroyed and the NRO reaction would not progress.

Base hydrolysis can be used to increase the resolution of the GRO method. If RNase hydrolysis is performed prior to the NRO reaction, base hydrolysis afterwards will be unnecessary.

Step 6: Selecting Hydrolyzed NRO-RNA with a Solid Support to Obtain a Highly Enriched and Purified Fraction of the NRO-RNA

The GRO method comprises selecting hydrolyzed NRO-RNA with a solid support to obtain a highly enriched and purified fraction of the hydrolyzed NRO-RNA. The solid support can be a bead support, column matrix, membrane support, biochip, microtiter plate or microfluidic device, all of which are well known in the art. In one embodiment, the isolated. NRO-RNA containing Br-UTP and α-³²P-CTP can, be hydrolyzed (in Step 5) to an average size of ˜100 bases. In Step 6, the hydrolyzed NRO-RNA can be bound to a solid support such as affinity purification beads (e.g., agarose) conjugated with a moiety that binds. BrU contained within the NRO-RNA. The moiety can be, for example, an antibody, an aptamer or a protein that reversibly binds BrU contained within the NRO-RNA.

Since the NRO-RNA is a small fraction of the RNA present in isolated nuclei, the method can employ specific isolation of NRO-RNA in order to reduce signal from RNA that was transcribed prior to the NRO reaction. In one embodiment, the method utilizes the nucleotide analog 5-Bromo-UTP (BrU) in place of UTP during the NRO reaction step and utilizes monoclonal or polyclonal antibodies that bind BrU-containing RNA to physically isolate and purify the NRO-RNA away from pre-existing RNA from nuclei. General methods for isolation and purification of tagged RNA using antibodies directed against the RNA tag are well known in the art.

In other embodiments, other U analogs known in the art can also be used. In such embodiments, binding partners known in the art to bind the U analog can be conjugated with the solid support.

Incorporation of BrdU into DNA or BrU into RNA has classically been used to identify locations of active transcription in whole cells by immunofluorescence of fixed tissue culture slides. Methods for affinity isolating NRO-RNA using BrUTP and an α-BrUTP antibody are known in the art (see, e.g., U.S. 2007/0141558 A1, Mar. 23, 2005, entitled Quantitative assay for detection of newly synthesized RNA in a cell-free system and identification of RNA synthesis inhibitors by Huang et al.; Pindolia, Kirit R.; Lutter, Leonard C. (2005) Purification and Characterization of the Simian Virus 40 Transcription Elongation Complex. Journal of Molecular Biology, 349(5): 922-932).

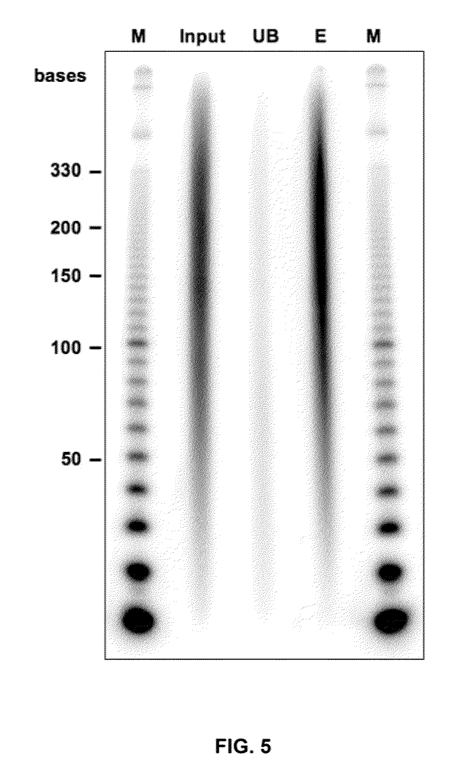

A mouse monoclonal anti-5-Bromo-deoxyUTP (α-BrdU) antibody known in the art (Santa Cruz Biotech, product # SC-32323-AC) cross reacts very well with BrU-containing RNA and can be used to physically isolate and purify the NRO-RNA away from pre-existing RNA from nuclei (FIG. 5). α-BrU antibodies can be conjugated to agarose beads and hydrolyzed NRO-RNA is applied to the beads. The beads are washed to remove non-specifically bound RNAs, and the bound RNA is removed by destruction of the α-BrU antibody through, e.g., SDS and DTT treatment.

In one embodiment, hydrolyzed NRO-RNA is triple-selected. To ensure that isolated RNA is highly enriched for NRO-RNA, the binding to the solid support, washing and elution of the RNA can be repeated twice more. When the NRO-RNAs are to be ligated directly to adapters, repeated binding to the solid support after the ligation steps is also important for removing excess linkers that did not participate in the ligations and will contaminate downstream procedures. After this triple-selection, the NRO-RNA can be >450,000-fold enriched and >99% pure.

FIG. 5 shows the results of binding and elution of base-hydrolyzed BrU-RNA to α-BrdU beads. In this embodiment, isolated NRO-RNA containing Br-UTP and α-³²P-CTP can be hydrolyzed to an average size of 100-150 bases and then bound to agarose beads that are conjugated with the α-BrdU antibody. The beads can be washed several times and then eluted. Equivalent amounts of each fraction can be run on a denaturing gel, e.g., an 8% denaturing PAGE gel, to assess the efficiency of bead binding.

Step 7: Enzymatically Repairing, Hydrolyzed NRO-RNA

The GRO method comprises enzymatically repairing hydrolyzed NRO-RNA (Step 7) before ligating the hydrolyzed NRO-RNA directly to adapter oligos that are compatible with any genomic assay platform (Step 8).

In one embodiment, the step of enzymatically repairing the hydrolyzed NRO-RNA comprises removing the 5′ cap. In a specific embodiment, removing the 5′ cap is accomplished through tobacco acid pyrophosphatase (TAP) treatment.

In another embodiment, the step of enzymatically repairing the hydrolyzed NRO-RNA comprises adding a 5′-P. In a specific embodiment, adding the 5′-P is accomplished through neutral T4 polynucleotide kinase (T4 PNK) treatment.

In another embodiment, the step of enzymatically repairing the hydrolyzed NRO-RNA comprises removing a 3′-P. In a specific embodiment, removing the 3′-P is accomplished through low pH T4 PNK treatment or by using other phosphatases well known in the art.

The ends of the hydrolyzed NRO-RNA are enzymatically repaired or altered to make them suitable substrates for the RNA ligase reactions. In one embodiment, the hydrolyzed NRO-RNA obtained from the first round of binding to and elution from the solid support can be treated with treated an enzyme to remove the 5-methyl guanosine cap, e.g., tobacco acid pyrophosphatase (TAP). The RNA is then treated with T4 Polynucleotide kinase (T4 PNK) at low pH to remove the 3′ phosphate. The 3′ phosphate can also be removed by treatment with alkaline phosphatases as known in the art. Finally, 5′ phosphates are added to the hydrolyzed products by further treatment with T4 PNK in neutral pH buffer in the presence of ATP. The NRO-RNA is now compatible with RNA ligases and the 5′ adapter oligo is added directly to the RNA with T4 RNA ligase I in Step 8 (below).

Step 8: Ligating Hydrolyzed NRO-RNA Directly to Compatible Adapter Oligos

The GRO method comprises ligating the hydrolyzed NRO-RNA directly to adapter oligos that are compatible with a genomic assay platform.

The ligating step is preferably done after one round of selecting the hydrolyzed NRO-RNA with the solid support. Otherwise, contaminating RNA will participate in the ligation rather than the nascent RNA. Step 7 does not need to be repeated.

Adapter oligos can be compatible with, for example, Illumina, SOLiD, 454 (Roche), or any other sequencing or array technologies. Compatible adapter oligos are well known in the art (see, e.g., Lau et al., Science (2001) 294:858-6)

Methods for adding adaptors to the hydrolyzed RNA are well known in the art (see, e.g., U.S. Pat. No. 6,544,736, Apr. 8, 2003, to Shimamoto et al., entitled Method for synthesizing cDNA from mRNA sample; U.S. Pat. No. 4,661,450, Apr. 28, 1987, to Kempe et al. entitled Molecular cloning of RNA using RNA ligase and synthetic oligonucleotides; U.S. Pat. No. 6,238,865, May 29, 2001, to Huang et al. entitled Simple and efficient method to label and modify 3′-termini of RNA using DNA polymerase and a synthetic template with defined overhang nucleotides; U.S. Pat. No. 5,688,670, Nov. 18, 1997, to Szostak et al., entitled Self-modifying RNA molecules and methods of making).

The GRO method can determine not only the location of transcribing polymerases with high resolution, but also the direction in which the polymerases are transcribing (strand specificity). To retain strand specificity of the isolated NRO-RNA molecules, distinct (i.e., having different sequences) oligonucleotide adapters are ligated to the 5′ end and 3′ ends of the RNA. Hydrolyzed RNA (e.g., based-hydrolyzed RNA), however, is not compatible with conventional single-stranded RNA ligase enzymes that add oligos to the 5′ and 3′ ends of RNA. Nucleic acid polymers are linked through a phosphate group at the 5′ end and a hydroxyl group at the 3′ end. The products of base hydrolysis, however, are RNA molecules with a hydroxyl group at the 5′ end and phosphate group at the 3′ end. In addition, NRO-RNA that originates near a transcription start site will also have a 5-methyl guanosine cap attached to the 5′ phosphate, which also makes the RNA incompatible with RNA ligase reactions.

In the previous step (Step 7) the ends of the hydrolyzed NRO-RNA are enzymatically repaired or altered to make them suitable substrates for the RNA ligase reactions. As described above, the NRO-RNA from the first round of bead binding/elution can be treated with tobacco acid pyrophosphatase (TAP) to remove the 5-methyl guanosine cap. Any enzyme known in the art to remove the cap structure can be used.

If hydrolyzed by base chemical treatment (base or divalent cation treatment), the RNA is then treated with T4 polynucleotide kinase (T4 PNK) at low pH to remove the 3′ phosphate. Alternatively, one can use a number of commercially available alkaline phosphatases to remove the 3′ phosphate. However, T4 PNK treatment is preferred, because the user can then proceed directly to the next step, rather than having to inactivate the phosphatase by extraction of the enzyme.

Finally, 5′ phosphates are added to the base hydrolyzed products by further treatment with T4 PNK in neutral pH buffer in the presence of ATP. The NRO-RNA is now compatible with RNA ligases and the 5′ adapter oligo is added directly to the RNA with T4 RNA ligase I.

The RNA can then be subjected a second round of binding to a solid support and elution followed by addition of the 3′ adapter oligo (e.g., repeat steps 6 and 8 without performing another round of step 7). A third round of selection by binding to a solid support can also be performed. Three rounds of selection by binding to solid support are preferable: for increasing the efficiency of the enzymatic reactions, for increasing the purity of the NRO-RNA, and for removing the excess 5′ and 3′ adaptor oligos, such that they do not interfere with downstream reactions.

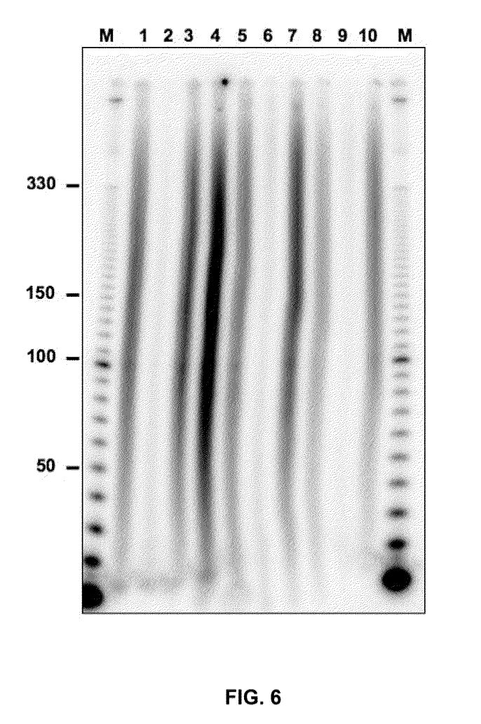

FIG. 6 shows an analysis of fractions from the major steps of the GRO method, which shows that the RNA remains intact throughout the steps of the method.

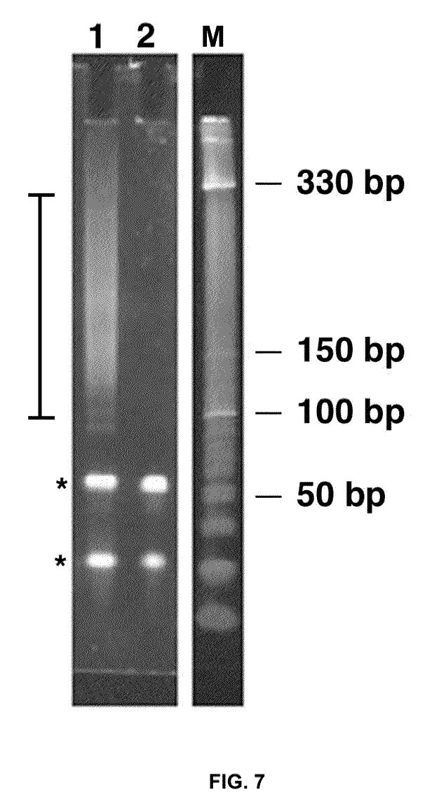

In one embodiment, the NRO-RNA is then reverse transcribed using an oligo that is complimentary to the 3′ adapter. NRO-cDNA second strand can then be made by DNA extension from a primer that is the DNA equivalent of the 5′ RNA adapter. The double-stranded NRO-cDNA can then be amplified (e.g., by PCR) with the same DNA oligos to produce a ‘NRO-library’ (FIG. 7).

FIG. 7. Shows an example of an amplified NRO-library cDNA. After the third elution the library was reverse transcribed amplified by 15 cycles of PCR, and then run on an 8% PAGE gel for purification away from the primers (*) Lane 1 cDNA library, Lane 2) No template control. Bracket indicates region cut from gel.

5.2 Modifications of the GRO Method

In one embodiment the steps of the GRO method described in Section 5.1 can be modified to adapt the method to identifying a transcription start site in the genome of a cell of interest. The method can comprise the steps of:

1) permeabilizing the cell of interest or isolating the nucleus from the cell of interest;
2) performing a nuclear run-on (NRO) reaction with the permeabilized cell or the isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration or duration of the NRO reaction;
4) isolating NRO-RNA from the NRO reaction;
5) hydrolyzing the NRO-RNA isolated from the NRO reaction to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA;
8) selecting capped NRO-RNAs through enzymatic enrichment by the oligo-capping method; and
9) ligating the hydrolyzed NRO-RNA to compatible adapter oligos.

In another embodiment, the GRO method can be modified to adapt the method to identifying an active site of an engaged RNA polymerase in the genome of a cell of interest comprising the steps of:

1) permeabilizing the cell of interest or isolating the nucleus from the cell of interest;
2) hydrolyzing RNA in the permeabilized cell or the isolated nucleus with an RNase;
3) performing a nuclear run-on (NRO) reaction with the permeabilized cell or the isolated nucleus, wherein a purifiable nucleotide analog is added to the NRO reaction;
4) optimizing the number of bases traveled by engaged polymerases for high resolution and low bias for nucleotide content of transcribed sequences by limiting a second nucleotide concentration or duration of the NRO reaction;
5) isolating NRO-RNA from the NRO reaction;
6) selecting hydrolyzed NRO-RNA with a solid support to obtain a highly enriched and purified fraction of the hydrolyzed NRO-RNA;
7) enzymatically repairing the hydrolyzed NRO-RNA by removing a 5′ cap from the NRO-RNA and adding a 5′-P to the NRO-RNA; and
8) ligating the hydrolyzed NRO-RNA to compatible adapter oligos.

According to this modification, the RNA is hydrolyzed by RNase prior to the NRO reaction. Then the GRO-method proceeds as normal, except that two steps are subsequently omitted: performing base hydrolysis after the NRO reaction and removing the 3′-P (e.g., through treatment with low pH T4 PNK) which would normally be done after removing the 5′ cap (e.g., through treatment with (TAP).

In another embodiment, the GRO method can be modified to adapt the method to map a site of co-transcriptional cleavage that delineates the 3′ end of an mRNA comprising the steps of:

1) permeabilizing the cell of interest or isolating the nucleus from the cell of interest;
2) performing a nuclear run-on (NRO) reaction with the permeabilized cell or isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration or duration of the NRO reaction;
4) isolating NRO-RNA from the NRO reaction;
5) optionally hydrolyzing the NRO-RNA isolated from the NRO reaction, to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA removing a 3′-P from the hydrolyzed NRO-RNA; and
8) ligating the hydrolyzed NRO-RNA to compatible adapter oligos.

In one embodiment, the modified method can comprise, after the step of ligating the hydrolyzed NRO-RNA to compatible adapter oligos, the step of amplifying the NRO-RNA.

In another embodiment, the modified method: can comprise the step of performing reverse transcription after the step of ligating the hydrolyzed NRO-RNA to compatible adapter oligos, wherein the ligating step comprises addition of a RNA oligomer to the 5′-end of the NRO-RNA and addition of an RNA oligomer to the 3′-end of the NRO-RNA.

5.3 Methods for Making the GRO Method Compatible with Massively Parallel Sequencing Platforms

The GRO method can be compatible with any platform that is used to survey the position of sampled sequences over entire genomes. Such platforms include, but are not limited to microarray, bead array, and massively parallel sequencing technologies. When the GRO method is made compatible with any sequencing technology it is referred to herein as ‘Global Run-On sequencing’ (GRO-seq), and when it is combined with microarray platforms it is referred to herein as ‘Global Run-On chip’ (GRO-chip).

To make GRO compatible with each of these technologies, different sets of 5′ and 3′ end adapters are ligated to the NRO-RNA during the steps described above. Methods for massively parallel sequencing of DNA are well known in the art (see, e.g., U.S. Pat. No. 6,013,445, Jan. 11, 2000, entitled Massively parallel signature sequencing by ligation of encoded adaptors; U.S. Pat. No. 5,695,934, Dec. 9, 1997, entitled Massively parallel sequencing of sorted polynucleotides).

Methods for microarray analysis are also well known in the art (see, e.g., Roche Nimblegen, http://www.nimblegen.com/, last visited Sep. 3, 2009).

There are currently three commonly used platforms for massively parallel sequencing of DNA available through Roche (454-sequencing), Applied Biosystems (SOLiD), and Illumina (Solexa 1G genome analyzer). Each sequencing platform uses a different methodology as well as different sequencing adapter oligos that can be attached to the ends of the isolated NRO-RNA during the steps described above. These different methodologies and adapter oligos are well known in the art.

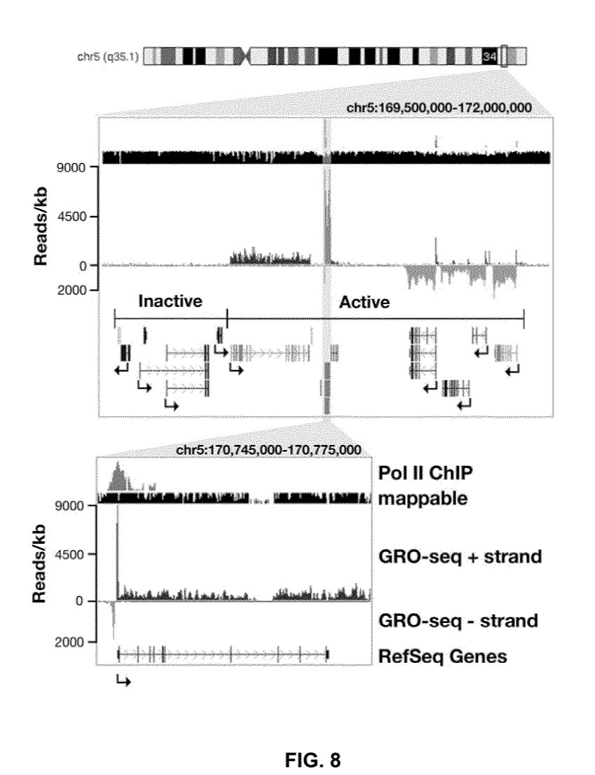

The amplified NRO-library can then be sequenced from what represents the 5′ end of the original RNA molecule using any sequencing method known in the art. The sequence reads are then mapped to a reference genome such that the exact position, orientation, and relative number of hits for each sequence are known. FIG. 8 shows an example of the data when mapped to the human genome and viewed in the UCSC genome browser available at http://genome.ucsc.edu (last visited Aug. 30, 2009). The data was obtained after sequencing ˜25,000,000 GRO library molecules as viewed in the UCSC genome browser. Shown is a 2.5 mb region on chromosome 5: 141,180,000-14,585,000 bp, showing GRO-seq hits aligned to the genome at 1 bp resolution, followed by an up-close view around the NPM1 gene (chr5: 170,745,000-170,775,000 bp). All data is displayed on the UCSC genome browser. Information track are from top to bottom as follows: Pol II ChIP (chromatin immunoprecipitation assay) results are shown in, mappable regions, GRO-seq hits on the plus strand (left to right, GRO-seq hits on the minus strand (right to left), RefSeq gene annotations.

5.4 Methods for Making GRO Compatible with Microarray Hybridization Platforms

To render the GRO method compatible with microarrays, DNA can be fluorescently labeled directly and hybridized to the array. For example, a phage promoter (T7) can be added to transcribe the DNA into RNA. This further amplifies the signal and at the same time, nucleotide analogs can be incorporated that are used for detecting the signal by fluorescence. Numerous phage promoters known in the art can be used for this, e.g., SP6 and T3.

In one embodiment, NRO-RNAs can be ligated to an oligo containing a T7 RNA polymerase promoter. The T7 promoter is added to the 5′ end and a DNA oligo that comprises a generic nucleic acid sequence that is not present in the genome of interest is ligated to the 3′ end to allow for reverse transcription and amplification as described above. The amplified NRO library is then transcribed in vitro by T7 RNA polymerase in the presence of nucleotide analogs called amino alkyl nucleotides. The resulting transcribed RNA is then labeled through a standard alkylation reaction that attaches either a fluorophore or biotin molecule to the RNA. The labeled RNA is then applied to the desired array and allowed to hybridize to sequences on the array.

For RNA that is labeled with a fluorophore, the level of hybridization to specific features on the arrays can be directly detected by standard methods of fluorescence detection. For RNA that is labeled with biotin, the RNA is mixed with a solution of streptavidin molecules that are conjugated to a fluorophore. Streptavidin binds very efficiently and specifically with the biotin-RNA, and detection on the array can be carried out by standard fluorescence detection. Other standard methods for nucleic acid labeling prior to hybridization to microarrays may also be used.

5.5 Considerations in Adapting the GRO Method for Various Genomic Platforms

In certain preferred embodiments of the GRO method, Steps 1-8 are carried out as described above in Section 5.1.

Currently available sequencing platforms vary in the number and lengths of sequence reads per run, reagent costs, and library preparation protocols. 454-sequencing, available from Roche, offers the longest reads (˜250 bases), but has the disadvantage of a low number of reads (˜3×105/run) and high reagent costs compared to the platforms offered by Illumina and Applied Biosystems. These two systems offer shorter reads (33-36 base's) but obtain larger numbers of reads per run (4×107 and 8×107 for Illumina and ABI, respectively). The greater depth of coverage afforded by high numbers of reads can provide more efficient quantification of nascent transcripts, and the shorter read lengths are sufficient for accurate mapping of the transcripts to genomes. This is important for coverage. It has been estimated that there are ˜90,000 active polymerases in HeLa cells: 15,000 Pol I, 65,000 Pol II, and 10,000 Pol (Faro-Trindade and P. R Cook, Biochemical Society Transactions (2006) 34, 1133-1137). Ensuring the detection of genes containing low levels of transcriptionally-engaged polymerases requires the sequencing of millions of run-on RNAs.

5.6 Uses for the GRO Method

The GRO method can be used to map the position, direction and level of transcriptional engaged RNA polymerase molecules throughout any sequenced genome under any condition. The GRO method can be used to generate a snapshot of the steady state transcription levels in cells. Current technologies analyze accumulated RNA levels under various conditions in order to identify transcription networks involved in particular processes. However, the level of RNA detected by these methods is a function of both the rate of synthesis and degradation of said RNA. The GRO method analyzes the rate of synthesis of RNAs, and thus provides a means to quantify the direct effect of cellular processes on transcription. Also, by comparing the rate of synthesis detected by the GRO method with the level of accumulated RNAs detected by other methods, one can identify genes that are regulated by accelerated or low rates of RNA turnover. By performing the method in cells under various treatments, one can comprehensively identify direct transcriptional outcomes of particular cellular processes ranging from response to stress, hormones, drugs, cell cycle progression, depletion of factors of interest, and the transcriptional changes associated with types of cancer.

5.7 Advantages of the GRO Method

Using the GRO method, high resolution of RNA polymerase location can be obtained as compared to other methods (mostly due to one nucleotide limitation and hydrolysis).

Furthermore, the GRO method is not limited to using radioactive uridine. Instead it a non-radioactive uridine derivative can be used.

The GRO method can be used to derive the directionality of RNA polymerase.

The GRO method is more sensitive, e.g., at least 1000× more sensitive, than a traditional NRO, and at least 100× more sensitive than a microarray.

Furthermore, with GRO, a read of active genes can be obtained within 2-3 min after cell treatment. Compared with 20-30+ minutes generally needed using any other method known in the art (owing to longer preparation and/or processing times), this is very rapid. At 2-3 minutes, initial gene activation can be observed that is in response to the initial cell treatment. By contrast, after 20-30 minutes, the gene activation measured by the other assays could be in response to events occurring after the initial cell treatment.

Whereas NRO only measures the activity of a few genes, GRO measures all actively transcribing genes.

In embodiments of GRO in which successive binding/purification steps (e.g., triple-selection) are used, sensitivity and accuracy are dramatically increased.

### 6. EXAMPLES

### 6.1 Example 1

### Development of GRO-seq in a Human Lung Fibroblast Cell Line

This example describes the development of the experimental conditions for the GRO method as well as the application of the method combined with sequencing technology (called GRO-seq) to a human lung fibroblast cell line, IMR90. GRO-seq has also been applied by us to a human breast cancer cell line (MCF7), a mouse embryonic stem cells, mouse embryonic fibroblasts, and Drosophila S2 cells, demonstrating that GRO-seq is a general method that can be used for analysis of various cell lines from various species (data not shown).

6.1.1 Background

Nuclear Run-On (NRO) assays have been used to measure the density of transcribing polymerases over specific targeted regions of the genome, and variations of the assay are capable of mapping the position of polymerases with high precision (P. Gariglio, J. Buss, M. H. Green, FEBS Lett. 44, 330 (1974); P. Gariglio, M. Bellard, P. Chambon, Nucleic Acids Res. 9, 2589 (1981); A. E. Rougvie, J. T. L is, Cell 54, 795 (1988); and E. B. Rasmussen, J. T. L is, Proc. Natl. Acad. Sci. U.S.A. 90, 7923 (1993). Traditionally, nuclei are isolated, endogenous nucleotides are removed by washing, and radionucleotides are added back allowing transcriptionally engaged polymerases to resume elongation. The incorporated radiolabel is restricted to sequences immediately downstream of the original position of the transcriptionally-engaged polymerase by keeping run-on reaction times short. The anionic detergent sarkosyl, which does not interfere with elongating polymerases, is often added to the nuclear run-on reaction to ensure that new transcription initiation events do not occur, and to remove physical impediments that can block elongation (A. E. Rougvie, J. T. L is, Cell 54, 795 (1988); D. K. Hawley, R. G. Roeder, J. Biol. Chem. 260, 8163 (1985). Thus all new transcription is produced by polymerases that are engaged at the time of nuclear isolation.

The RNA is then isolated and hybridized to filters containing genes or gene regions of interest. These measurements have been shown to represent the level of transcriptionally-engaged polymerase on genes at the time of nuclei isolation, and have also been used to identify Pol II that is paused at the 5′ ends of genes as well as the distance Pol II travels beyond the 3′-ends of genes prior to termination J. L is, Cold Spring Harb. Symp. Quant. Biol. 63, 347 (1998); I. Faro-Trindade, P. R. Cook, Biochem. Soc. Trans. 34, 1133 (2006); N. J. Proudfoot, Trends Biochem. Sci. 14, 105 (1989); and N. Gromak, S. West, N. J. Proudfoot, Mol. Cell. Biol. 26, 3986 (2006).

Previous attempts at scale-up have hybridized radiolabeled NRO RNAs to cDNA probes spotted on macroarrays to analyze how steady state transcription of genes relates to mRNA accumulation (M. Schuhmacher et al., Nucleic Acids Res. 29, 397 (2001); J. Garcia-Martinez, A. Aranda, J. E. Perez-Ortin, Mol. Cell. 15, 303 (2004). These methods can give reasonable approximations for steady state transcription levels for some genes; however, they suffer from low sensitivity, lack of whole genome coverage, and no resolution within gene regions. Whole genome coverage is important for detection of novel transcription units as well as transcripts that are not present in cDNA libraries.

The lack of resolution of cDNA arrays is of concern since genes that have a promoter-proximal paused Pol II and do not produce full-length transcripts will produce detectable signal that does not reflect actual levels of full-length transcription of those genes (L. J. Schilling, P. J. Farnham, Nucleic Acids Res. 22, 3061 (1994). In addition, the distribution of transcribing polymerases within genes provides information on how a particular gene is regulated. When this information is combined with knowledge of promoter DNA sequences, transcription factor binding sites, and nucleosomes and their modifications, it can further understanding of how these elements cooperate to specify distinct transcriptional outcomes.

6.1.2 Development of GRO-seq

6.1.2.1 Incorporation of Br-UTP by Nuclear RNA Polymerases

Given that the NRO-RNA represents a small fraction of the total RNA in nuclei (see below), analysis of NRO-RNA with conventional genomic platforms requires specific isolation of this RNA. To adapt nuclear run-ons for a global analysis, it was reasoned that a nucleotide with an affinity purifiable tag could be added to the run-on reaction. Thus the incorporation and purification efficiencies were tested as described below.

It was first tested whether 5-Bromo-UTP (BrUTP) could be efficiently incorporated into RNA by nuclear RNA polymerases by also incorporating a radioactive nucleotide (α³²P-CTP) in a run-on time course experiment. Consistent with previous results (F. J. Iborra, A. Pombo, D. A. Jackson, P. R. Cook, J. Cell. Sci. 109 (Pt 6), 1427 (1996), addition of Br-UTP allowed incorporation at ˜80% efficiency compared with UTP, and approximately 10 fold over the control that lacked both UTP and Br-UTP (FIG. 2).

In FIG. 2, polymerases were run-on in nuclei supplemented with Sarkosyl, ATP, GTP, α-³²P-CTP and UTP (open diamonds), Br-UTP (closed triangles), or no UTP (open circles), Separate reactions were setup for each time point and the reactions were stopped at 5, 10, 25 or 45 min. The RNAs were isolated, and the radioactivity incorporated was assayed by scintillation counting.

These radiolabeled RNAs made in the presence of Br-UTP bind very well to anti-Br-deoxy-U beads, which cross-reacts well with BrU (FIG. 5) (see below).

FIG. 5 shows binding and elution of base-hydrolyzed BrU-RNA to α-BrdU beads. Isolated RNA from a nuclear run-on containing Br-UTP and α-³²P-CTP was base hydrolyzed to an average size of 100 bases, and then bound to agarose beads that are conjugated with an antibody specific for α-BrdU. The beads were washed several times and then eluted. Equivalent amount of each fraction were run on an 8% denaturing. PAGE gel to assess the efficiency of bead binding.

Although BrU is sometimes used as a mutagen, sequencing clones from GRO-seq libraries indicated the misincorporation rate by nuclear RNA polymerases is low. The propensity of BrU to cause misincorporation during reverse transcription was also tested by comparing sequencing results of cDNA clones that were generated from RT reactions that contain a BrU or U RNA template of known sequence. The results showed that there was no appreciable level of misincorporation by reverse transcriptase when BrU is incorporated into the RNA template (data not shown). BrU was thus chosen as the affinity tagged nucleotide for further development of the assay.

6.1.2.2 Control of Resolution for GRO-seq

The GRO-seq method can be used to isolate and obtain a high resolution and unbiased map of all RNAs as they are being transcribed. High resolution requires that run-on distances are kept short, whereas unbiased mapping requires efficient incorporation of the affinity-tagged nucleotide analog into all RNAs. Nucleotide concentrations were titrated during the run-on step and defined the minimum distance for library preparation as the lowest concentration that allows maximum binding of the run-on RNAs to beads. To determine the length of the run-on transcription, nuclei were first pre-treated with RNase in order to trim the nascent RNAs (D. A. Jackson, F. J. Iborra, E. M. Manders, P. R. Cook, Mol. Biol. Cell 9, 1523 (1998). RNA polymerases can protect the nascent RNA from 15-25 bases upstream from the active site W. Gu, M. Wind, D. Reines, Proc Natl Acad Sci USA 93, 6935 (1996); M. L. Kireeva et al., Mol Cell 18, 97 (2005). The RNase activity was then removed through extensive washing and treatment with RNase inhibitor. The distance polymerases run-on was then controlled by titrating limiting concentrations of CTP.

To identify locations of RNA polymerase II (Pol II), the distance transcribed by polymerases in the presence of α-amanitin and actinomycin-D was examined. α-amanitin is an efficient inhibitor of Pol II, but works much less effectively on Pal III, and is completely innocuous for Pol I transcription (D. A. Jackson, F. J. Iborra, E. M. Manders, P. R. Cook, Mol. Biol. Cell 9, 1523 (1998). Actinomycin-D, when added to cells prior to nuclei isolation, primarily inhibits Pol I.

By comparing the length of nascent transcripts produced from RNase treated nuclei and in the presence of inhibitors, the distance Pol II transcribed under various limiting nucleotide concentrations was deduced (FIG. 3).

FIG. 3 shows control of nuclear run-on distance by limiting nucleotide concentration. Nuclei were pre-treated with RNase to reduce the nascent RNA to ˜20 nucleotides, washed, and then allowed to run-on in separate reactions containing a α-³²P-CTP and cold CTP for a total of 0.65 μM (Lane 2), 1 μM (lane 3), 5 μM (lane 4) or 25 μM (lane 5). Non-RNase treated nuclei supplemented with 1 μM total CTP were used as a control (Lane 1). Cells were treated with Act-D and nuclei were treated with α-amanitin.

Analysis of the efficiency of bead binding under similar conditions is shown in FIG. 4. FIG. 4 shows bead binding efficiency in response to [CTP] titration. Nuclear run-ons were performed as described in FIG. 3, but without pre-treatment with RNase. Run-on RNAs from each sample were base hydrolyzed and bound to equivalent amounts of beads. The bound and unbound fractions were monitored for radioactivity by scintillation counting. The percent bound (y-axis) was calculated relative to input fractions and is displayed relative to the concentration of CTP in the reaction (x-axis).

The analysis in FIG. 4 showed that with nuclei from IMR90 cells, 1 uM CTP was sufficient to allow near maximum bead binding. This corresponded to a run-on extension of ˜80-100 nucleotides (FIG. 3), which was the average length of the RNAs (˜100-120 nucleotides) subtracted by the length of RNAs protected by the polymerase (˜20 nucleotides). 1 uM CTP was therefore considered the optimum concentration for these nuclei.

In non-RNase treated nuclei (which were used for creating the NRO-library), base hydrolysis of the nascent RNAs to an average size that was equal to the length of the run-on transcripts then resulted in a final mapping resolution of approximately half this distance. Base hydrolysis of the RNA improved the resolution of this assay by severing the extended portions of the nascent RNA transcript that contained the nucleotide analog from distal regions that were transcribed prior to the run-on reaction. In this study, Pol II was allowed to run-on approximately 80-100 bases, thus the resolution was estimated to be 40-50 bp from the location of the polymerase active site at the time of the assay.

6.1.3 Yield, Enrichment and Purity of Nascent RNA after Triple Selection

High sensitivity and specificity is desired in any genomic assay in order to decrease both false negative and false positive results. These parameters require that both the yield and enrichment of run-on RNAs be high relative to contaminant RNAs.

6.1.3.1 Enrichment by Tracking Radiolabeled NRO-RNAs

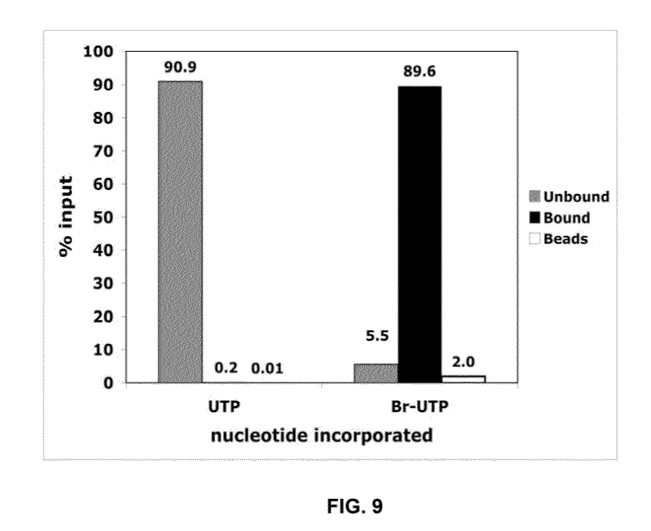

To assess the specificity and efficiency of the purification, the enrichment of the nascent RNAs was measured by incorporating a radiolabeled nucleotide (α-³²P-CTP) in run-on reactions performed in the presence of either UTP or Br-UTP. Quantification of the bound and unbound fractions from each reaction by scintillation counting showed that the enrichment by this method is ˜450 fold for a single round of bead binding (FIG. 9). FIG. 9 shows the specificity of α-BrdU beads. Run-ons were performed in the presence of either UTP or Br-UTP and handled as described previously. RNAs from each fraction were quantified by scintillation counting.

Successive enrichment could not be examined because the amount of radioactivity in the UTP-RNA was below the limit of detection in the bound fraction after binding to a new set of beads.

To assess whether contaminant RNA was able to cross-hybridize with BrU-RNA, a bead binding was performed with α³²P-CTP radiolabeled, UTP-containing RNA in the presence of non-radioactive, BrU RNA. Under these conditions the level of radioactivity in the bound fraction was the same as CTP-labeled samples containing only UTP, suggesting that cross-hybridization was negligible.

6.1.3.2 Measurement of Enrichment and Purity by RT-qPCR

Since the amount of radiolabeled NRO-RNA measured in the above experiments was a minor fraction of the total RNA isolated from nuclei, a significant amount of contaminant RNA could still exist after triple selection.

There was 50 μg in the starting pool and 300 ng in the elution from the third round of bead binding for the Br-UTP samples. Spiking controls were added consisting of multiple small (˜100 base) RNAs that were in vitro transcribed in the presence of either UTP or Br-UTP. The cDNAs used for in vitro transcription were reverse transcribed and amplified from Arabidopsis thaliana total RNA. U-RNAs were added in 10-fold dilutions from 1×10¹⁰-1×10⁷ copies and a BrU-RNA was added at 1×10⁷ copies.

After triple selection, reverse transcription followed by quantitative PCR (RT-qPCR) was carried out on the final elution for each RNA. The BrU-RNA was present at 50% relative to input, and all U-RNAs were at or below background for the assay. The lowest amount of the input detectable was 1:10,000, therefore non-BrU RNAs could be present at 1:10,000^{th} relative to the starting amount. This corresponded to 5 ng since 50 μg of nuclear RNA was the starting amount. Since the final elution contains 300 ng of RNA, U-RNA represented 1.6% of the final mass, corresponding to >98% purity for BrU-RNA. Subsequent libraries were constructed that contained the U-RNAs and BrU-RNA as spike-in controls. Quantification of the number of reads from each control revealed that the RNA is enriched >450,000 fold, making the final cDNA libraries >99% pure.

In addition to the above results, several computational analyses suggested that the NRO-RNA libraries were highly enriched for NRO-RNA relative to accumulated RNAs. First, an estimation of background was determined by binning reads in 500 kb windows genome wide. The distribution of windows with the lowest densities fit a Poisson distribution corresponding to spreading 2-3% of the aligned reads randomly over the mappable portion of the genome, agreeing well with the above experimental results and suggesting that background for the assay approaches 0.04 hits on a single strand per 1 kb.

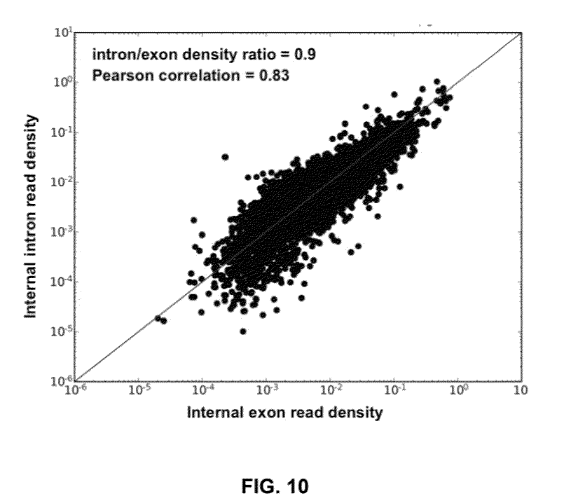

Second, transcription was detected in regions of transcription units that were not present in fully processed mRNAs, including introns and regions beyond the site of nascent RNA cleavage and polyadenylation. The ratios of read density within introns vs. exons was 0.9 (Pearson correlation=0.83), and was not significantly different from 1 (P=0.71, FIG. 10).

FIG. 10 is a comparison of GRO-seq read density in exon versus intron. The scatter plot shows the density of GRO-seq reads within introns (y axis) versus exons (x-axis) for each RefSeq gene. Axes are in log10 scale. Only internal exons and introns were used in the analysis to avoid inflation of signal due to promoter-proximal pausing or build up of polymerases that can occur near the 3′-end of genes.

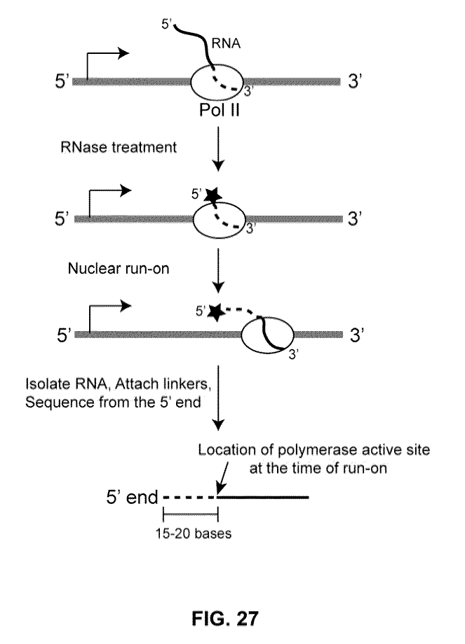

Third, known gene deserts ranging from 0.6 Mb to 3 Mb have an average density of reads on both strands together of 0.07 hits/1 kb, which also agreed well with the experimental and computational analyses of background (Table 1 in FIG. 28). Table 1 is a background calculation in gene deserts. The indicated large intergenic spaces were analyzed for the number of GRO-seq reads on either strand and for the number of mappable bases.

6.1.4 Overview of GRO-seq Method

This section describes the overall method to accompany FIG. 1. In this example, the GRO method was combined with massively parallel sequencing technology from Illumina to produce GRO-seq. The methods section below gives a detailed description of the steps involved. Nuclei isolation and run-on reactions were performed using standard protocols with the exception that 5-Bromo-UTP was used in place UTP, and the concentration of CTP was adjusted to 1 μM to keep the run-on distance to ˜100 nucleotides (see above). α-³²P-CTP was also used as a tracer in order to follow the purification steps and analyze the products on denaturing PAGE.

RNA was isolated and base hydrolyzed to the desired size. RNA fragments were then isolated by binding to anti-deoxy-BrU beads to select against accumulated nuclear RNAs, washed several times, and eluted from the beads. Because base hydrolysis of RNA leaves a molecule with a 5′-hydroxyl and a 3′-phosphate, neither of which are substrates for ligation of adapter oligos, the RNA ends were repaired. First, the RNAs were treated at low pH with tobacco acid pyrophosphatase to remove 5-methyl guanosine caps (E. B. Rasmussen, J. T. L is, Proc. Natl. Acad. Sci. U.S.A. 90, 7923 (1993) and then were treated at low pH with T4 polynucleotide kinase (PNK) to remove the 3′-phosphate (V. Cameron, O. C. Uhlenbeck, Biochemistry 16, 5120 (1977). The pH was then raised and the RNA was treated again with PNK, except of the reaction now contained ATP, to add a 5′-phosphate. An adapter was then added to the 5′-end with T4-RNA ligase and the RNA was bound to anti-deoxy-BrU beads to remove excess linkers and further enrich the RNA. This process was then repeated for the addition of a 3′-adapter. The affinity-enriched RNAs were then reverse transcribed, amplified, and PAGE purified.

Analysis of a fraction of each step by denaturing polyacrylamide gel electrophoresis (FIG. 6) showed that the RNA remained largely intact throughout the procedure.

FIG. 6 shows denaturing PAGE analysis of fractions from GRO-seq library preparation. Lanes: 1) Input, 2) Unbound-1, 3) Elution-1, 4) After TAP-PNK treatment, 5) 5′ adapter ligation, 6) Ubound 2, 7) Elution 2, 8) 3′ adapter ligation, 9) Unbound 3, 10) Elution 3.

FIG. 7 shows an example of an amplified NRO-library cDNA. After the third elution the library was reverse transcribed amplified by 15 cycles of PCR, and then run on an 8% PAGE gel for purification away from the primers (*) Lane 1 cDNA library, Lane 2) No template control. Bracket indicates region cut from gel.

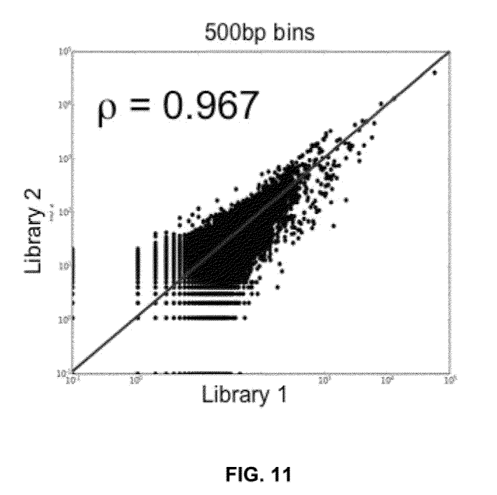

After amplification and PAGE purification, the library appeared to be, on average, 100 bases in length (˜190 base-90 base adapters). A known amount of the library was re-amplified to determine the primer efficiency from which the original complexity of the cDNA library could be extrapolated. In the two libraries constructed in this study, complexities of 1×10⁹ molecules were obtained prior to amplification. 50 molecules were also cloned and sequenced by conventional methods to verify the size and to ensure the quality of the library before massively parallel sequencing on the Illumina 1G genome analyzer. Analysis of the correlation of read densities throughout the genome from two biological replicates indicated that the GRO-seq method is highly reproducible (FIG. 11).

FIG. 11 shows correlation of GRO-seq biological replicates. GRO-seq transcript reads were mapped to the genome and unique hits were binned in 500 bp windows. Of the 6,160,849 windows, 3,458,076 windows had no hits in each replicate. The replicates show a correlation coefficient of 0.967 (Spearmann correlation). Thus correlation of the read densities between the two replicates produced in this study showed that replicates agreed remarkably well

### 6.2 Example 2

### Application of GRO-seq in a Human Lung Cell Fibroblast Cell Line, IMR90

6.2.1 Introduction

This example demonstrates that the GRO-seq method can be used to map the position, amount, and orientation of transcriptionally-engaged RNA polymerases genome-wide. These measurements provide a snapshot of genome-wide transcription and directly evaluate promoter-proximal pausing on all genes. Nuclear run-on RNAs are subjected to large-scale parallel sequencing and mapped to the genome. The example shows that peaks of promoter-proximal polymerase reside on ˜30% of human genes, transcription extends beyond pre-mRNA 3′ cleavage, and antisense transcription is prevalent. Additionally, most promoters have an engaged polymerase upstream and in an orientation opposite to the annotated gene. This divergent polymerase is associated with active genes, but does not elongate effectively beyond the promoter. These results demonstrate that the interplay between polymerases and regulators over broad promoter regions dictates the orientation and efficiency of productive transcription.

Nuclear run-on assays (NRO) were used to extend nascent RNAs that were associated with transcriptionally-engaged polymerases under conditions where new initiation is prohibited. To specifically isolate NRO-RNA, a ribonucleotide analog (BrUTP) was added to BrU tag nascent RNA during the ‘run-on’ step (FIG. 1). The length of the incorporated bases was kept short and the NRO-RNA was chemically hydrolyzed into short fragments (˜100 bases) to facilitate high-resolution mapping of the polymerase origin at the time of assay. BrU-containing NRO-RNA was triple selected through immuno-purification with an antibody that is specific for this nucleotide analog, resulting in at least 10,000-fold enrichment of NRO-RNA pool that was determined to be >98% pure. A NRO-cDNA library was then prepared for sequencing from what represents the 5′-end of the fragmented RNA molecule using the Illumina 1G high-throughput sequencing platform. The origin and orientation of the RNAs, and therefore the associated transcriptionally-engaged polymerases was documented genome-wide by mapping the reads to the reference human genome.

6.2.2 Methods

6.2.2.1 Isolation of Nuclei

Isolation of nuclei was carried out as described in L. J. Strobl, D. Eick, Embo J 11, 3307 (1992), with several modifications. 15 cm² plates of IMR90 cells (˜6×10⁶ cells at 80% confluency) were washed directly on the plate 3× with ice cold PBS. 10 ml of ice cold swelling buffer (10 mMTris-cl pH7.5, 2 mM MgCl2, 3 mM CaCl2) was added and allowed to swell on ice for 5 min. Cells were removed from the plate with a plastic cell scraper, transferred to a 15 ml conical, and pelleted for 10 min at 4° C. at setting 3 on an IEC clinical centrifuge. Cells were resuspended in 1 ml of lysis buffer (swelling buffer+0.5% Igepal, +10% glycerol+2 units/ml SUPERase In (Ambion), and gently pipetted up and down 20 times using a p1000 tip with the end cut off to reduce shearing. The volume was brought to 10 ml and nuclei pelleted at setting 4 on an IEC clinical centrifuge. The nuclei were washed and pelleted once in Lysis buffer, resuspended in 1 ml Freezing buffer (50 mM Tris-CL pH 8.3, 40% glycerol, 5 mM MgCl2, 0.1 mM EDTA), and transferred to a 1 ml tube. Nuclei were pelleted at 1000×g, and resuspended in 100 μl of Storage Buffer/5×10⁶ nuclei.

6.2.2.2 NRO-RNA Library Construction

Construction of a NRO-library for sequencing involved the run-on reaction, base hydrolysis, immuno-purification, end repair, 5′- and 3′-adapter ligation, amplification, and PAGE purification.

6.2.2.3 NRO Reaction

5×10⁶ IMR90 nuclei (100 ul) were mixed with an equal volume of reaction buffer (10 mM Tris-Cl pH 8.0, 5 mM MgCl2, 1 mM DTT, 300 mM KCL, 20 units of SUPERase In, 1% sarkosyl, 500 uM ATP, GTP, and Br-UTP, 2 μM CTP and 0.33 μM α-³²P-CTP (3000 Ci/mmol). The reaction was allowed to proceed for 5 min at 30° C., followed by the addition of 23 μl of 10×DNAseI buffer, and 10 μl RNase free DNase I (Promega). Proteins were digested by addition of an equal volume of Buffer S (20 mM Tris-Cl pH 7.4, 2% SDS, 10 mM EDTA, 200 ug/ml Proteinase K (Invitrogen), followed by incubation at 55° C. for 1 hour. RNA was extracted twice with acid Phenol: chloroform, and once with chloroform, and precipitated at a final concentration of 300 mM NaCl, with 3 volumes of −20° C. ethanol. The pellet was washed in 75% ethanol before resuspending in 20 μl of DEPC-treated water.

6.2.2.4 Base Hydrolysis of RNA

Base hydrolysis was performed on ice by addition of 5 μl 1M NaOH and incubated on ice for 30 min. The reaction was neutralized by addition of 25 μl 1M Tris-Cl pH 6.8. The reaction was then run twice through a p-30 RNase-free spin column (BioRad), according to the manufacturer's instructions. Before moving on to the immuno-purification, DNA was further removed by another digestion with RNase-free DNaseI for 10 min at 37° C., and the reaction stopped by addition of 10 mM EDTA.

6.2.2.5 Immuno-Purification of Br-U RNA

Anti-deoxyBrU beads (Santa Cruz Biotech) were blocked in 0.5×SSPE, 1 mM EDTA, 0.05% Tween, 0.1% PVP, and 1 mg/ml ultrapure BSA (Ambion). NRO-RNAs were heated to 65° C., added to 100 μl beads in 500 μl of binding buffer (0.5×SSPE, 1 mM EDTA, 0.05% Tween), and allowed to bind 1 hour while rotating. The beads were washed once in low salt buffer (0.2×SSPE, 1 mM EDTA, 0.05% Tween), twice in high salt buffer, 0.5% SSPE, 1 mM EDTA, 0.05% Tween, 150 mM NaCl), and twice in TET buffer (TE+0.05% Tween). The Br-U RNA was then eluted 4×125 μl of Buffer E (20 mM DTT, 300 mM NaCl, 5 mM Tris-Cl pH 7.5, 1 mM EDTA, and 0.1% SDS). The RNAs were then extracted and precipitated as above,

6.2.2.6 End Repair

Enriched RNAs were resuspended in 20 μl DEPC-treated water and incubated with 2.5 μl Tobacco acid pyrophosphatase (TAP, Epicentre Biotechnologies), 1×TAP buffer, and 1 μl SUPERase Inhibitor in a final volume of 30 μl at 37° C. for 1 hour. 1 μl of Polynucleotide Kinase (PNK, NEB), and 0.5 μl of 5 mM MgCl2 was then added and the reaction continued for 30 min. 20 μl PNK buffer, 2 μl 100 mM ATP, and 145 μl water, and 1 μl PNK was then added and the reaction continued for another 30 min. 90 μl water and 10 μl 500 mM EDTA was then added, followed by extraction and precipitation of the RNA.

6.2.2.7 Adapter Ligations

For adapter ligations the RNA was resuspended in 8.5 ul, and incubated with 2.5 μl of either the 5′- or the 3′-adapter oligo (Small RNA Isolation Kit, Illumina), 1 μl SUPERase In, 2 μl RNA ligase-1 buffer, 5 μl 50% PEG 8000, and 1.5 μl of T4 RNA ligase-1 (NEB). The reactions were incubated on the lab bench for 4 hours. After both the first and second adapter ligations, the RNAs were enriched over anti-deoxy-BrU beads as described above.

6.2.2.8 Reverse Transcription and Amplification and PAGE Purification of NRO-RNA Libraries

The RNAs were reverse transcribed (otherwise according to the manufacturer's specifications) in two separate 10 μl reactions, with 0.5 μl 100 uM RT-Primer (Illumina Small RNA Isolation Kit), and 1 μl SIII reverse transcriptase (Invitrogen), at 44° C. for 15 min, followed by 52° C. for 45 min. The RNAs were degraded by addition of RNase cocktail (Ambion), and RNase H (Ambion) and amplified 15 cycles with Phusion high fidelity DNA polymerase (Finnzymes) using the PCR primers specified by Illumina. The NRO-cDNA libraries were then run on a non-denaturing 1×TBE, 8% acrylamide gel and cDNAs greater than 90 nucleotides were excised from the gel and eluted by incubating in TE+300 mM NaCl overnight while rotating. The library was then extracted, precipitated, and then sent to Illumina for sequencing on the 1G Genome Analyzer.

6.2.2.9 Data Analysis

Alignment of GRO-seq reads to the human genome. Two independent biological replicates were submitted for sequencing at Illumina. Library 1 was sequenced on three channels and yielded 13,818,931 total reads while library 2 was sequenced on two channels and yielded 9,389,058 reads. All reads were 33 bases long. Alignments to the hg18 assembly of the human genome were performed with the Eland alignment tool from Illumina. 5,316,960 full length reads from library 1 aligned uniquely to the human genome and 4,459,581 full length reads from library 2 aligned uniquely to the human genome. Alignments allowed up to two mismatches per sequence to account for sequencing errors and SNPs between the IMR90 cell line and the sequenced genome.

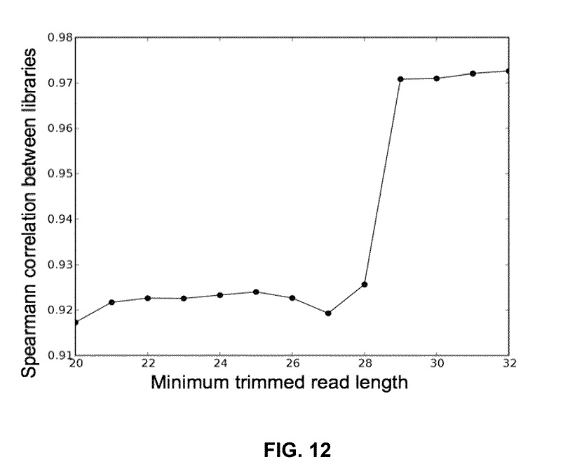

To increase the coverage of the libraries, one base was trimmed iteratively from the 3′ end of reads that did not align uniquely and checked if it now aligned uniquely at the reduced length. Trimming was done from the 3′ end, because the quality score for reads was highest at the 5′ end and lowest in the 3′ end, and because it was possible that some of the amplified library was shorter than the 33 bases sequenced. Analysis of the correlation between the two libraries as a function of trimming extent showed that 29 bases was the preferable minimum length to be included (FIG. 12).

FIG. 12 shows a plot interlibrary correlation versus read trimming. Reads that did not align uniquely were trimmed by one base at the 3′ end and realigned to the genome in an iterative process. The Spearmann correlation between the two libraries is shown as a function of the minimum length of the reads included in the libraries. Because the correlation drops when 28 mers are included, all analyses were performed with only 29 mers and longer.

Alignments were done to the full (non-repeat masked) human genome. While unique alignments can be achieved in repeat masked sequences, we analyzed the number of reads mapping to such repeat masked sequences to be sure they were trust worthy. With the exception of rRNA repeats, the density of alignments to repeat regions mirrored the average overall density of surrounding regions, suggesting that they were indeed accurate. The rRNA repeats, however, had an average density roughly five orders of magnitude above the average genome-wide level. Since rRNA is the most abundant mature RNA in the cell, it was the major non-NRO RNA contaminant in the purifications, and thus all alignments to rRNA repeats in the genome were removed. These steps increased the total number of reads aligned to the genome to 5,800,577 for library 1 and 4,950,956 for library 2, for a total of 10,751,533 unique alignments. Since sequencing was performed from the 5′ end of the BrU purified NRO RNA, the 5′ coordinate of each read was used as the position of engaged polymerase for all subsequent analyses.

Identifying mappable bases in the genome. To assess the fraction of the genome where reads could be expected to align, all unique 32 base sequences from both strands of the hg18 assembly were identified. This was a total of 2,414,845,175 32-mers per strand from a total possible 3,080436,051 per strand. A ‘mappable’ or ‘unmappable’ base refers to the 5′ base of a given mappable or unmappable 32-mer. All calculations of read densities in subsequent analyses were relative to these mappable bases.

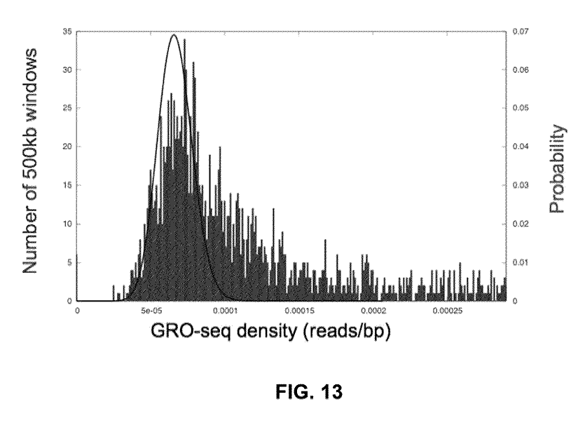

Background calculation from low-density windows. To assess the background GRO-seq density, the genome was divided into 500 kbp windows and the density of hits in each window was calculated. The distribution of low-density windows is described by placing 3% of the total GRO-seq reads randomly on the mappable portion of the genome (FIG. 13).

FIG. 13 shows a background calculation by low-density windows. After aligning reads to genome, the density of GRO-seq hits was assessed in 500 kb windows. Shown is a histogram of the lowest density windows and the solid line is a Poisson distribution with a mean given by placing 3% of all GRO-seq reads at random throughout the mappable portion of the genome.

The theoretical curve is described by

$$p ( x ) = \frac{{λ}^{x * l} {e}^{- λ}}{( x * l ) !}$$

where x is the density of reads on both strands per base pair, l is the window size (500 kb), and λ is the background density of reads (in units of reads/bp).

$$λ = \frac{f * {N}_{reads}}{{L}_{mappable}}$$

f is the fraction of all reads that are from background (0.03 in FIG. 13), N_{reads} is the total number of reads aligning to the genome (10,751,533) and L_{mappable} is the total number of mappable 32-mers in the genome summed over both strands (4,829,690,350).

Background calculation from gene deserts. Sixteen separate ‘gene deserts’ were identified where most GRO-seq alignments should represent background. These regions ranged in size from roughly 500 kb to nearly 7 Mb. The details of the coordinates of these gene deserts and the number of GRO-seq hits are in Table 1 (FIG. 28).

Calculation of gene activity. Gene activity was defined as NIL where N is the number of coding strand GRO-seq reads from +1 kb (relative to the TSS) to the end of each gene, and L is the number of mappable bases in this region. The significance of a given gene's activity level was determined by the probability of observing at least N reads in an interval of length L from a Poisson distribution of mean l=0.04 hits/kb (the background density of the libraries).

$$p = {\sum }_{n = N}^{∞} \frac{{( λ * L )}^{n} {e}^{- λ * L}}{n !}$$

If the probability was less than 0.01, the gene was called active. The first kilobase of each gene was omitted to better gauge the density of polymerase that actively elongates through the gene and to avoid over-counting from the increased density of paused polymerase in the 5′ end of the gene. All analyses were done with the complete RefSeq gene list for the hg18 assembly of the human genome reduced to include only genes at least 3 kb in length so that the measurement of GRO-seq density in the body of the gene would be robust.

Correlation of GRO-seq densities with microarray expression data. The previous expression microarray work (T. H. Kim et al., Nature 436, 876 (2005) had been performed on the Affymetrix U133Plus2 array. To correlate the GRO-seq data with this expression array data, the original array data was downloaded from the supplementary material of that paper and the knownToRefSeq and knownToU133Plus2 tracks from the UCSC genome browser were used to map RefSeq genes to probe IDs. The analysis of the array data was performed as in the original paper (T. H. Kim et al., Nature 436, 876 (2005). That is, a probe had to be present or absent in both replicates to be called present or absent. If all probes mapping to a particular gene were absent, then the gene was absent and if any probes mapping to a particular gene were present then, the gene was present. All other genes were considered ambiguous and removed from subsequent analyses.

Identification of promoter proximal peaks. The exact position of many TSSs is not precisely annotated and many promoters do not have a single well defined TSS (P. Carninci et al., Nat. Genet. 38, 626 (2006). Therefore, to identify the peak of promoter proximal coding strand GRO-seq reads, each annotated TSS 1 kb upstream and downstream was tiled around in 50 by windows, shifting by 5 bp. The number of coding strand reads and the number of mappable bases was counted in each window. The significance of the density was calculated in each window by comparing to the background density of 0.04 reads/kb in a manner similar to how gene activity significance was calculated (see above). The most significant window was chosen as the promoter proximal peak, and if multiple windows had the same significance, then the most 5′ of these windows was chosen. If the promoter proximal peak had a p value less than 0.001, the gene was identified as having a significant promoter proximal activity. To identify the divergent peak, a similar approach was used but tiling was done +/−1 kb from the identified promoter proximal peak and only reads on the noncoding strand were counted. The same p value cutoff of 0.001 was used to classify genes as having a significant peak of divergent transcription.

Identification of paused genes. Significantly paused genes were identified by using the Fisher exact test to compare the density of reads in the sense strand promoter proximal peak to the density of reads in the body of the gene as compared to a uniform distribution of all these reads based on the number of mappable bases. A p value cutoff of 0.01 was used to call significantly paused genes.

Extending peaks to transcribed regions. To measure how far the significant promoter proximal peaks could be extended into transcribed regions, the 3′ most read was identified within the peak (in a strand specific manner), and d(n), the distance from the current read to the n^{th} downstream read on the same strand, was calculated. If this distance was less than the cutoff distance, the 3′ boundary of the peak was extended to this n^{th} read and the process was repeated by shifting one read downstream. This process was continued until the peak could no longer be extended. The value of n used in: this analysis was 5 and the length cutoff was 2.5 kb.

Correlation of GRO-seq and ChIP-chip data. The previous ChIP-chip data was reported for positions relative to the hg16 assembly of the human genome (T. H. Kim et al., Nature 436, 876 (2005). The UCSC liftOver tool was used to convert these coordinates to the hg18 assembly. To assess GRO-seq levels around the TAF1 peaks identified in the previous work, either the GRO-seq density of the associated gene for the transcript-matched promoters or 1 kb upstream and downstream for the novel promoters were examined. For the transcript-matched promoters, gene activity values and significance were calculated as described above. For the novel promoters, the total number of reads on both strands and the number of mappable bases were counted. To identify significant transcription, a p value cutoff of 0.01 was used when comparing to the probability of obtaining that number of reads or more from a Poisson distribution with a rate of ˜0.08 reads/kb because both strands were being counted.

6.2.3 Results and Discussion

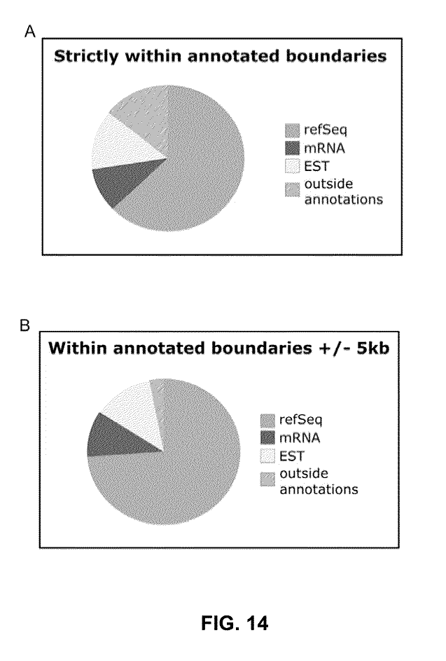

The results presented in this example demonstrate the application of the GRO method. FIGS. 8 and 12-24 display the typical results obtained. In total, ˜2.5×10⁷ 33 bp reads were obtained from two independent replicates (see above) prepared from primary human lung fibroblast nuclei (IMR90), of which ˜1.1×10^{7 (}44%) mapped uniquely to the human genome. Most reads (85.8%) aligned on the coding strand within boundaries of known RefSeq genes, human mRNAs, or expressed sequence tags (ESTs) (FIG. 14).

FIG. 14 shows GRO-seq reads relative to gene annotations. The fraction of reads aligning to the coding strand and strictly within the annotated boundaries (A) or within the annotated boundaries expanded by 5 kb (B). Reads were first mapped to RefSeq genes, then unmapped reads were mapped to Human mRNA, then reads that were still unmapped were mapped either to Human ESTs or outside annotations.

The number of transcriptionally active genes was determined using an experimentally and computationally determined background of 0.04 reads/kb. 6,882 (68%) of RefSeq genes were found to be active (P<0.01) compared to 8,438 active genes found by a microarray experiment performed in the same cell line (T. H. Kim et al., Nature 436, 876 (2005), reflecting, in part, the added sensitivity of sequencing platforms (M. Sultan et al., Science 321, 956 (2008). Examination of several large regions shows that GRO-seq differentiated between transcriptionally active and inactive regions in large chromosomal domains (FIG. 8). In addition, a generally low, but significant (P<0.01 relative to background) level of antisense transcription was detected for 14,545 genes (58.7% of genes in the genome).

FIG. 8 shows an example of the data when mapped to the human genome and viewed in the UCSC genome browser available at http://genome.ucsc.edu (last visited Aug. 30, 2009). The data was obtained after sequencing ˜25,000,000 GRO library molecules as viewed in the UCSC genome browser. Shown is a 2.5 mb region on chromosome 5: 141,180,000-14,585,000 bp, showing GRO-seq hits aligned to the genome at lbp resolution, followed by an up-close view around the NPM1 gene (chr5: 170,745,000-170,775,000 bp). All data is displayed on the UCSC genome browser. Information track are from top to bottom as follows: Pol II ChIP (chromatin immunoprecipitation assay) results are shown in, mappable regions, GRO-seq hits on the plus strand (left to right, GRO-seq hits on the minus strand (right to left), RefSeq gene annotations.

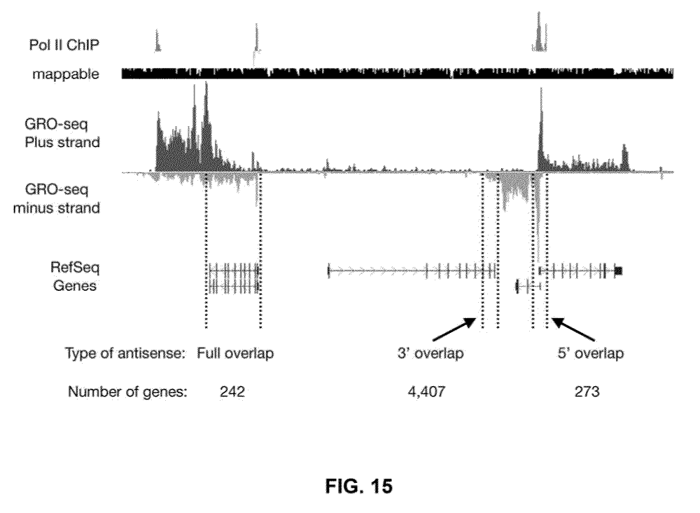

FIG. 15 shows the identification of antisense transcription GRO-seq. Three representative loci that show three types of antisense transcription identified previously by others, and presently in this study. The number of occurrences of (A) 5′-overlapping, (B) 3′-overlapping (convergent), and (C) fully overlapping antisense transcription is 273, 4,407, and 242, respectively.

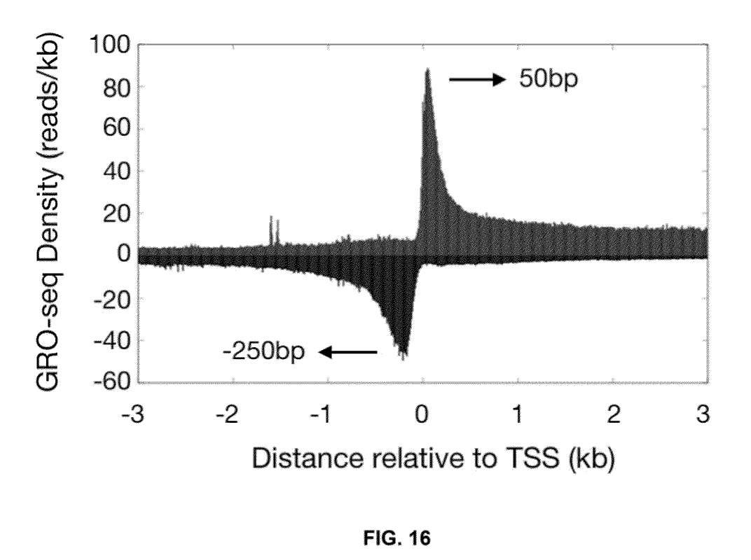

Aligning the GRO-seq data relative to RefSeq Transcription Start Sites (TSSs) showed that the highest density of reads peaked near the TSS in both the sense (˜50 bp) and antisense (˜−250 bp) directions (see below) (FIG. 16).

FIG. 16 shows Alignment of GRO-seq hits to TSSs (A) GRO-seq hits aligned to Ref-seq TSSs in 10 bp windows in both the coding (black) and non-coding (dark gray) directions relative to the direction of gene transcription.

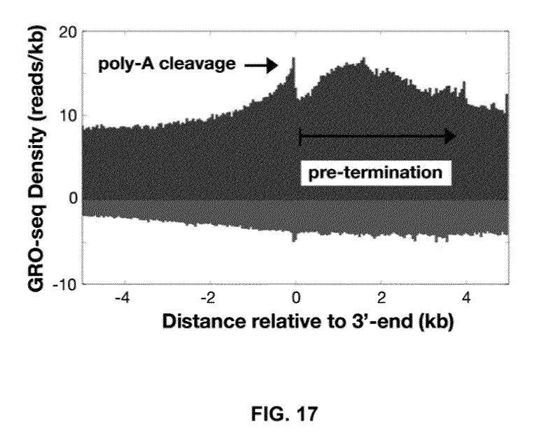

Alignment of GRO-seq reads to annotated 3′-ends of genes revealed a broad peak that was maximal at approximately +1.5 kb and extended greater than 10 kb downstream of poly-A sites (FIG. 17). This peak distance was consistent with previous and recent estimates (N. J. Proudfoot, Trends Biochem. Sci. 14, 105 (1989); Z. Lian et al., Genome Res. (2008). A small peak followed by a sharp drop off was observed at the site of polyadenylation likely representing the known 3′-cleavage prior to polyadenylation of the RNA (N. Proudfoot, Curr. Opin. Cell Biol. 16, 272 (2004).

FIG. 17 shows the alignment of GRO-seq hits to transcript ends. Two peaks are observed when aligning GRO-seq hits to the 3′-ends of genes. The first represents creation of a new 5′-end of the nascent RNA that results cleavage of the RNA at the poly-A site. The second peak at ˜+1 kb downstream represents slowing down of polymerases as they near termination which releases them from the DNA template.

6.2.3.1 Comparison of GRO-seq to Microarray Expression Data

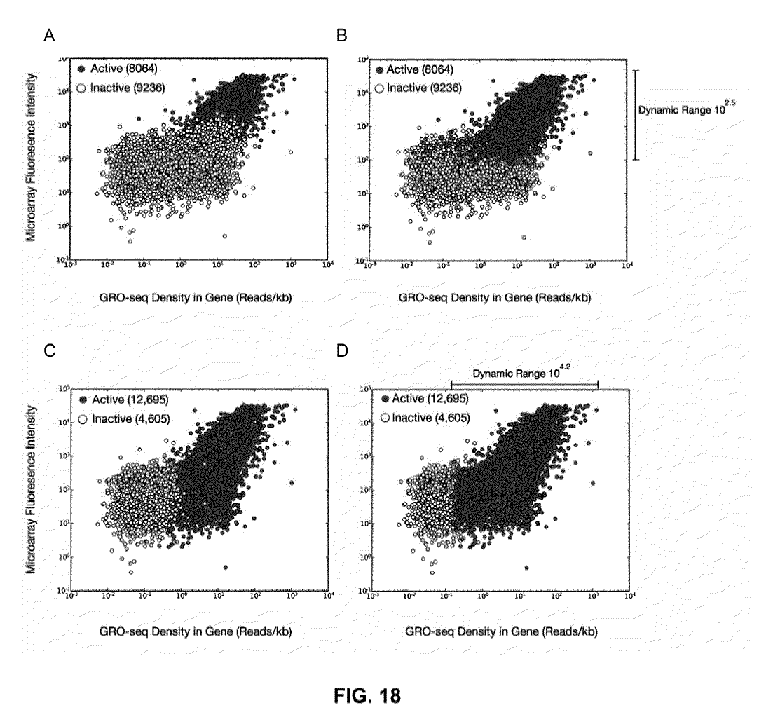

GRO-seq transcript densities in the sense orientation within gene regions were compared to the microarray expression data available for this cell line (. T. H. Kim et al., Nature 436, 876 (2005). First, microarray expression values plotted against GRO-seq densities revealed that accumulated, fully processed mRNA levels generally correlated with steady state transcription of genes obtained by GRO-seq (FIG. 18). However, GRO-seq densities had a wider dynamic range that extended below the limit of detection by microarray (compare FIG. 18A, B with 18C, D). That is, the microarray signal plateaus in the lower range leading to an increased fraction of inactive genes, whereas GRO-seq is able to call many of these gene transcriptionally active.

FIGS. 18A-D demonstrate the increased dynamic range and sensitivity in calling active genes obtained by the GRO-seq method as compared to microarray gene expression data. FIG. 18A is a scatter plot of microarray signal (y-axis) against GRO-seq signals (GRO-seq density, hits/base, x-axis) within genes. Inactive genes are white circles; active genes are dark gray. The range for which genes can be called significantly active is shown to the right (D), or top (E) for microarray hybridizations or GRO-seq, respectively. Note that GRO-seq, as performed here, has a wide dynamic range that results in increased sensitivity when identifying active genes.

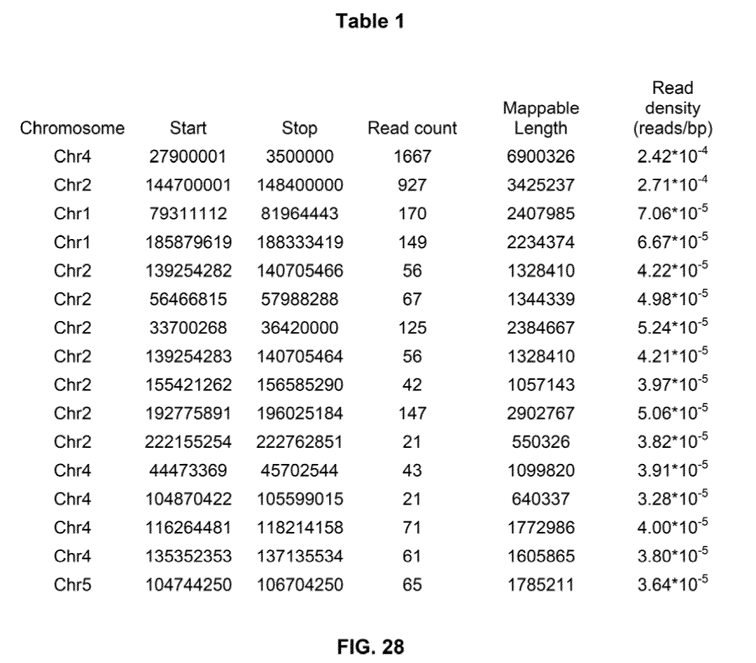

To gauge the increase in sensitivity, genes called absent or present by microarray were compared to genes that could be called active or inactive by GRO-seq. For a gene to be called active by GRO-seq, the density within the downstream portions of genes had to be significantly above background (P<0.01). The first 1 kb was excluded from the analysis to avoid signals produced by promoter-proximal paused polymerases (see methods). When considering all RefSeq genes, 16,882 genes (68%) were classified as active by GRO-seq. When considering the genes covered by probes on the microarray, 16,858 genes were called active by GRO-seq, while only 8,438 were called active by microarray hybridization (FIG. 18, Table 2 in FIG. 29).

Active gene calls for GRO-seq spanned more than four orders of magnitude, whereas microarray experiments were restricted to approximately 2.5 orders of magnitude (FIG. 18). The increased number of active genes in the GRO-seq analysis could be attributed to the increased sensitivity of sequencing technologies versus hybridization methodologies (B. Wold, R. M. Myers, Nat. Methods 5, 19 (2008); B. T. Wilhelm et al., Nature (2008), and possibly due to the fact that nascent RNA libraries may be enriched for rare or unstable transcripts relative to highly accumulated RNAs. The expression of several genes that were called active by GRO-seq but inactive by microarray by RT-qPCR was validated (see below).

6.2.3.2 Validation of GRO-seq Gene Activity by RT-qPCR

Transcripts that were regulated by post-transcriptional mRNA turnover were identified by comparing mRNA levels to GRO-seq densities. A highly stable transcript would be expected to have a high level of mRNA expression compared to the GRO-seq density within the corresponding gene, while unstable transcripts would be expected to have higher GRO-seq densities relative to mRNA expression level. By comparing GRO-seq with expression microarray data, candidates were identified as stable or unstable transcripts by searching for genes that were microarray active: GRO-seq inactive or microarray inactive: GRO-seq active, respectively.

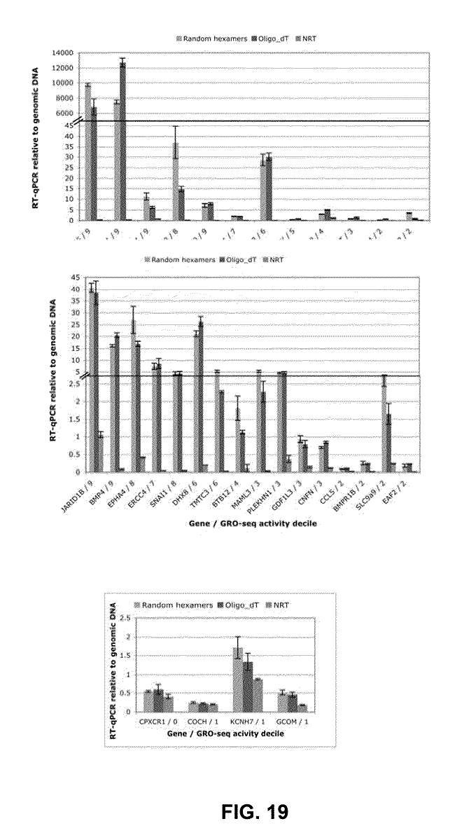

Several of these genes to genes that were found to be active in both assays were compared by performing RT-qPCR. The genes from each class were ranked into deciles of gene activity as determined from the GRO-seq density within gene bodies. Genes were chosen from a range of activity deciles to validate. The results showed that all genes tested that were called active by GRO-seq were detected by RT-qPCR after priming the reverse transcription with either random hexamers or oligo-dT to extents that generally mirrored their level of GRO-seq transcription (FIG. 19.).

FIG. 19 shows RT-qPCR validation of GRO-seq levels. Genes that were active by microarray and GRO-seq (A), inactive by microarray—active by GRO-seq (B), and active by microarray but not by GRO-seq (C) were analyzed by RT-qPCR. Reverse transcription was performed with random primers, or oligo-dT, and compared to a known amount of genomic DNA. No reverse transcription reactions are shown. Error bars represent standard error of the mean, n=3.

In addition, genes that were not detected by the microarray had similar RT-qPCR levels as those that were not detected by the arrays. These results verify GRO-seq as a general and sensitive method for detecting active genes and suggest that many genes were not detected by the microarray due to insufficient sensitivity or incorrect probe design. Two genes (COL1A1, IGFBP5) may be highly stabilized transcripts because they were called active by both microarray and GRO-seq, but were detected by microarray at much higher levels than other genes that were inactive by microarray but had similar GRO-seq densities.

Accumulated mRNA levels and GRO-seq density on the body of genes generally showed a strong concordance in IMR90 cells (FIGS. 18, 19). The relatively limited dynamic range and sensitivity of the microarray data may have caused some less stable RNAs to be missed. Also, classes of genes that are regulated by mRNA stability might be more readily detectable in response to changing environments (M. Schuhmacher et al., Nucleic Acids Res. 29, 397 (2001); J. Garcia-Martinez, A. Aranda, J. E. Perez-Ortin, Mol. Cell. 15, 303 (2004). Comparison of GRO-seq to RNA-seq data should also improve the efficiency of identifying mRNAs that are regulated by mRNA turnover rates (B. Wold, R. M. Myers, Nat. Methods 5, 19 (2008); B. T. Wilhelm et al., Nature (2008); U. Nagalakshmi et al., Science 320, 1344 (2008); A. Mortazavi, B. A. Williams, K. McCue, L. Schaeffer, B. Wold, Nat. Methods (2008).

6.2.3.3 Characteristics of Gene Transcription Revealed by GRO-seq

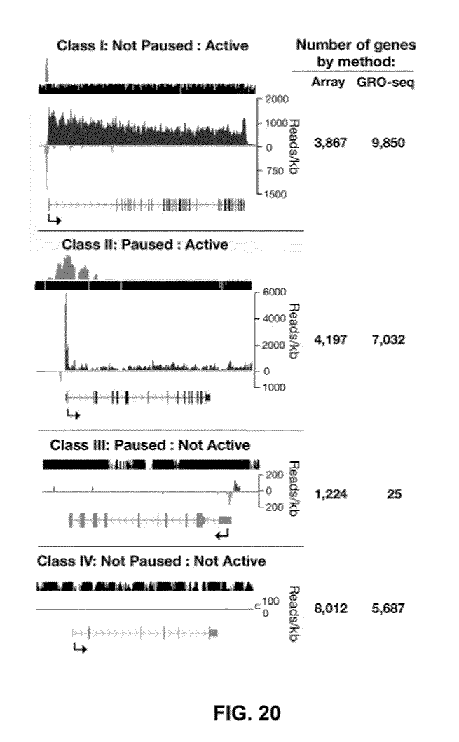

To identify all genes that show a peak of engaged Pol II that was characteristic of promoter-proximal pausing, it was assessed whether each gene showed significant enrichment of read density in the promoter-proximal region relative to the density in the body of each gene (See methods). The ratio of these densities is called the pausing index (G. W. Muse et al., Nat. Genet. 39, 1507 (2007); J. Zeitlinger et al., Nat. Genet. 39, 1512 (2007); see methods) and significant pausing indices ranged from 2 to 10³. 7,522 genes had a significant enrichment of GRO-seq reads within the defined promoter region relative to the body of genes (P<0.01), representing 28.3% of all genes (41.7% of active genes). Comparison of paused genes to either microarray expression or GRO-seq data revealed four classes of genes: not paused and active, II) paused and active, III) paused and not active, and IV) inactive (not paused and not active) (FIG. 20).

FIG. 20 shows a comparison of pausing with gene activity. Four classes of genes are found when comparing genes with a paused polymerase and transcription activity either by microarray or GRO-seq density in the downstream portions of genes. An example of each class is shown, with tracks shown in the UCSC genome browser as in FIG. 8. The gene names, pausing index, and P value, from top to bottom, respectively, are as follows: TR10, 1.1, 0.62; FUS, 41, 2.8×10⁻⁴³; IZUMO1, 410, 7.6×10⁻³; and GALP (which has no reads and therefore no pausing index). The number of genes represented in each class is shown to the right.

Class III was severely depleted when GRO-seq was used to classify gene activity, likely owing to a matter of sensitivity, since the few genes left within this class had very low signal at their promoters. Therefore, the overwhelming majority of genes with a paused polymerase also produced significant transcription throughout the gene, albeit often to levels not detectable by expression microarrays. A recent comparison of Pol II ChIP-seq data to RNA-seq also supports the view that virtually all genes that are bound by Pol II produce full length transcripts (M. Sultan et al., Science 321, 956 (2008).

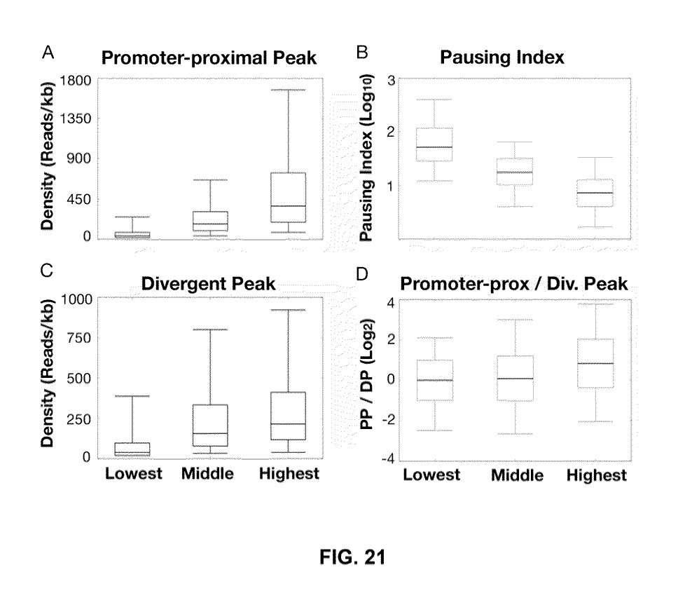

The density of polymerases within the promoter-proximal region generally correlated with the level of gene activity when all genes (FIG. 21A), or only genes with a paused polymerase were considered (data not shown). Whereas nearly all paused genes show significant full-length activity by GRO-seq, the pausing index inversely correlated with gene activity (FIG. 21B). Considering that pausing was observed when Pol II enters a pause site faster than the rate of escape from pausing (L. J. Core, J. T. L is, Science 319, 1791 (2008), this inverse correlation showed that highly transcribed, but paused genes, are controlled, at least in part, by increasing the rate at which Pol II escapes the pause site and enters productive elongation.

FIG. 21. Shows the correlation of promoter-proximal transcription patterns with gene activity. (A-D) Box plots (each showing the 5^{th}, 25^{th}, 50^{th}, 75^{th}, and 95^{th} percentiles) that show the relationship of Promoter-proximal (PP) sense peaks (top left), divergent peaks (DP) (bottom left), Pausing indices (top right) and PP/DP ratios (bottom right) to the top, middle and bottom deciles of gene activity. All deciles are significantly different from each other: P<10⁻⁹ for all comparisons except between the lowest and middle deciles in D (P<10⁻³). (E) ChIP profiles of Pol II and GRO-seq aligned to TSSs. (F) ChIP profiles of H3ac and H3K4me2 and GRO-seq aligned to TSSs.

6.2.3.4 Pausing and Gene Activity

As gene activity increases, it is expected that the occupancy of Pol II at promoters will also increase. This was borne out in ChIP data, as well as in the GRO-seq data presented here. FIG. 21B shows that GRO-seq density within promoter-proximal regions generally increased as the density of reads in the body of genes increased. However, pausing indices have an inverse correlation with gene activity. This relationship could reflect that highly expressed genes either did not experience pausing, or they transitioned through pausing faster, allowing more polymerase to enter into productive elongation.

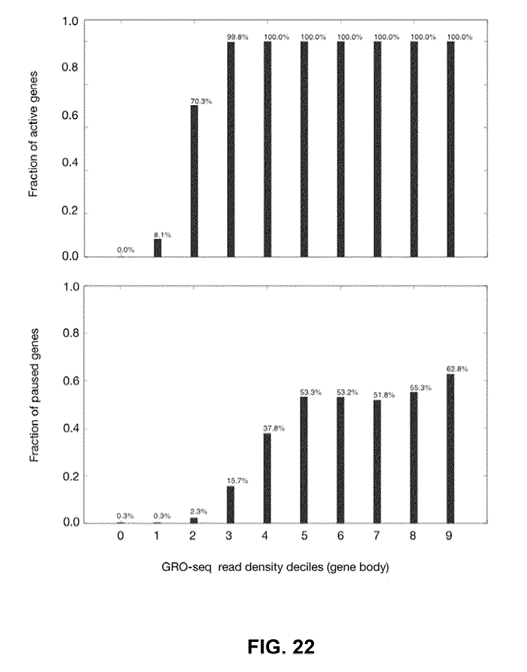

When the fraction of paused genes was examined according to gene activity deciles (FIG. 22), the fraction of paused genes was found to increase with increasing gene activity and represented 63% of the highest decile of gene transcription.

FIG. 22 shows the fraction of paused genes and active genes by gene activity decile. The percentage of significantly active (A) and significantly paused (B) genes in each docile of gene activity is shown. This result, in combination with the inverse correlation between gene body density and pausing indexes, indicated that highly active genes, relative to genes with lower activity, not only recruited more polymerase and stimulated faster pause site entry rates, but they also increased pause site escape to a greater extent to account for these profiles.

6.2.3.5 Gene Ontology of Paused Genes

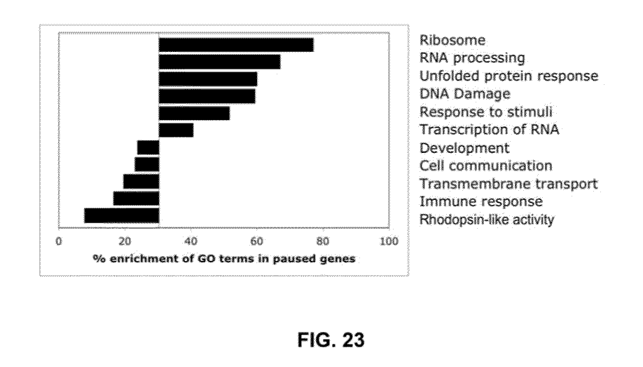

Significantly paused genes are enriched with biological processes such as cell cycle regulation, stress response, and protein biosynthesis (ribosomal proteins), and are de-enriched for developmentally regulated genes (FIG. 23).

FIG. 23 shows the gene ontology of paused genes. The bar plots shows the summary of enriched and de-enriched gene ontology (GO) terms of significantly paused genes. The Y-axis is set to 28.3%. GO terms that are enriched in paused genes are to the right of the axis, and GO that are de-enriched are to the left. All terms are significant (p<10⁴⁰).

Although previous studies identified developmentally regulated genes as enriched in the paused class (G. W. Muse et al., Nat. Genet. 39, 1507 (2007); J. Zeitlinger et al., Nat. Genet. 39, 1512 (2007); M. G. Guenther, S. S. Levine, L. A. Boyer, R. Jaenisch, R. A. Young, Cell 130, 77 (2007), these studies used either embryonic stem cells, an embryonic-derived cell line, or developmentally staged Drosophila embryos. The differences likely reflected the more differentiated state of the primary fibroblasts used in this study.

6.2.3.6 GRO-seq Results for Known Paused Genes

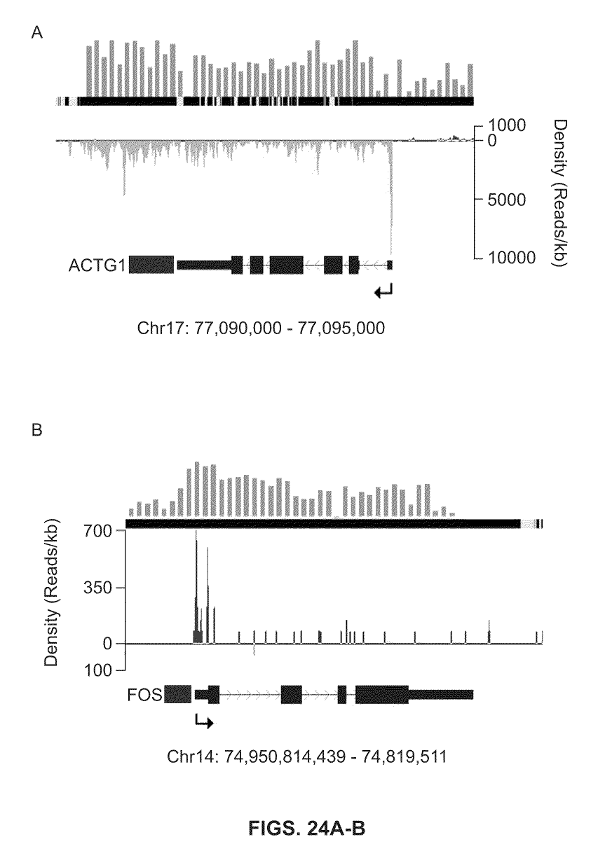

Several human genes have been shown to have a high level of transcriptionally engaged Pol II at the 5′-end relative to the downstream portions either by traditional NRO-hybridization assays, or by potassium permanganate footprinting. The genes include MYC (A. Krumm, T. Meulia, M. Brunvand, M. Groudine, Genes Dev 6, 2201 (1992); L. J. Strobl, D. Eick, Embo J 11, 3307 (1992)., FOS (J. Fivaz, M. C. Bassi, S. Pinaud, J. Mirkovitch, Gene 255, 185 (2000), DHFR (C. Cheng, P. A. Sharp, Mol Cell Biol 23, 1961 (2003), ACTG1 (γ-Actin) (C. Cheng, P. A. Sharp, Mol Cell Biol 23, 1961 (2003), and HSPA1A (HSP70) (S. A. Brown, A. N. Imbalzano, R. E. Kingston, Genes Dev 10, 1479 (1996). The first four genes exhibit a pattern consistent with pausing (FIG. 24) and are called significantly paused by the GRO-seq analysis. The human genome has two nearly identical copies of the HSP70 gene and could not be analyzed, because reads mapping to multiple locations were removed before any analysis was performed.

6.2.3.7 Divergent Transcription at Promoters

Another feature of the GRO-seq profiles around transcription start sites was the robust signal from an upstream, divergent, engaged polymerase. RNAs generated by these divergent polymerases can be identified at low levels when small RNAs are isolated from whole cells (Seila et al. Divergent Transcription from active promoters (19 Dec. 2008) Science 322 (5909), 1849). These divergent polymerases could not be accounted for by the 10% of known bidirectional promoters that are less than 1 kb apart (N. D. Trinklein et al., Genome Res. 14, 62 (2004). 13,633 genes (55% of all genes, 77% of active genes) displayed significant divergent transcription within 1 kb upstream of sense-oriented promoter-proximal peaks (P<0.001), indicating that the number of bidirectional promoters exceeded even the highest estimates (P. Kapranov et al., Science 316, 1484 (2007); A. Rada-Iglesias et al., Genome Res. 18, 380 (2008). However, since the majority of these promoters produced mRNAs in only one direction (see below), this new class of promoters was referred to as divergent. Although the top 10% of active genes had, on average, a slightly larger promoter-proximal than divergent peak (FIG. 21-4D), levels of divergent transcription generally correlated with both the promoter-proximal signal and the transcription level of the associated gene (FIG. 21-4C). Thus, divergent transcription was a mark for active promoters.

FIG. 21. Shows the correlation of promoter-proximal transcription patterns with gene activity. (A-D) Box plots (each showing the 5^{th}, 25^{th}, 50^{th}, 75^{th}, and 95^{th} percentiles) that show the relationship of Promoter-proximal (PP) sense peaks (top left), divergent peaks (DP) (bottom left), Pausing indices (top right) and PP/DP ratios (bottom right) to the top, middle and bottom deciles of gene activity. All deciles are significantly different from each other: P<10⁻⁹ for all comparisons except between the lowest and middle deciles in D (P<10⁻³). (E) ChIP profiles of Pol II and GRO-seq aligned to TSSs. (F) ChIP profiles of H3ac and H3K4me2 and GRO-seq aligned to TSSs.

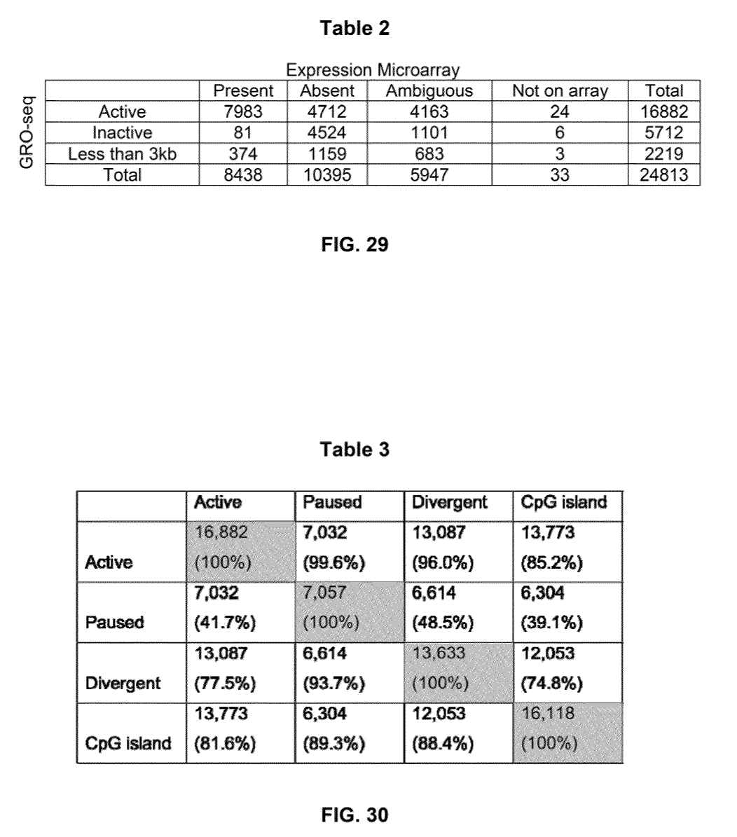

Gene activity, pausing, and divergent transcription correlated with each other and with promoters containing a CpG island. These four characteristics co-occurred significantly more often than would be expected by chance (P<10⁻⁵²) (Table 3 in FIG. 30). Previous mapping of capped mRNA transcripts has shown that at CpG island, promoter initiation occurs broadly over hundreds of base pairs (P. Carninci et al., Nat. Genet. 38, 626 (2006). The GRO-seq method described in this example shows that polymerases initiate and accumulate on this large class of promoters, in both orientations.

Table 3 (FIG. 30) shows pairwise correlations between Gene Activity, Pausing, Divergent transcription, and CpG island promoters. Four qualities of individual genes were found to significantly co-occur by pairwise tests. The four qualities were significant levels of gene activity, significant levels of pausing, a significant peak of divergent transcription, and having a CpG island-type promoter. The criteria for gene activity, pausing, and divergent transcription are described in the methods. To define whether a given promoter had a CpG island the CpG Islands track was downloaded from the UCSC Genome Browser. If there was an annotated CpG island within 1 kb of a given TSS, the gene was classified as having a CpG island-type promoter. The percentages listed in the Table are the fraction of genes from the category on the left that are also in the category on the top.

6.2.3.8 Comparison of GRO-seq to Existing ChIP Data

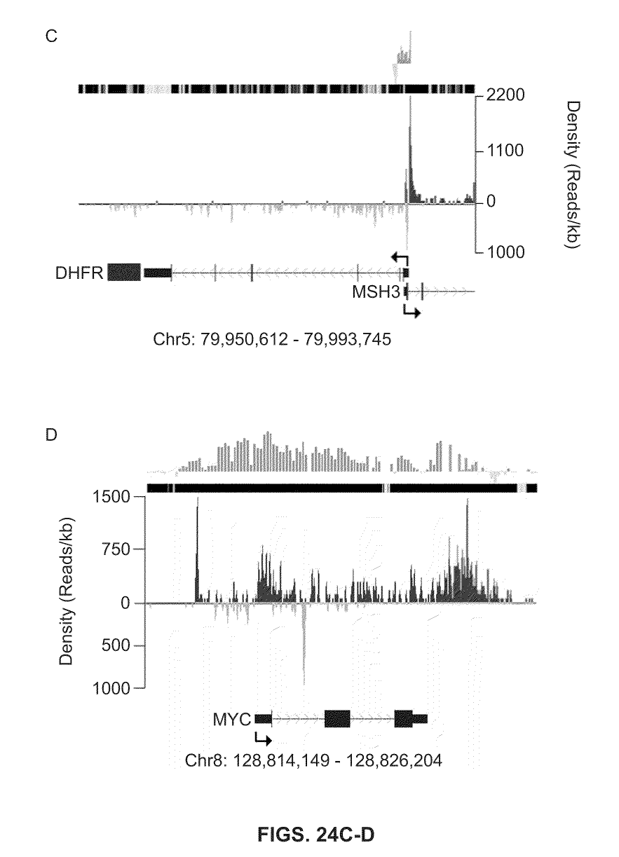

Does existing ChIP-chip data show any indication of the divergent peak of polymerase (T. H. Kim et al., Nature 436, 876 (2005)? Manual inspection of a number of genes and comparison with composite profiles aligned to TSSs showed that the Pol II ChIP peak at promoters was clearly accounted for by the two divergent peaks uncovered by GRO-seq (FIG. 25).

FIG. 23 shows ChIP profiles of Pol II and GRO-seq sense (S) and antisense (AS) strand reads aligned to TSSs, and ChIP profiles of H3ac and H3K4me2 and GRO-seq aligned to TSSs.

Higher resolution ChIP-seq data in different cell lines has identified Pol II upstream of promoters that are likely representative of the divergent promoters identified by GRO-seq (M. Sultan et al., Science 321, 956 (2008). Additionally, active promoters are typically marked by histone modifications such as di- and tri-methylation of H3-Lysine 4 (H3K4me2, H3K4me3) as well as acetylation of histone H3 and H4 (H3ac, H4ac). These modifications show a bimodal distribution around TSSs, with the trough representing a nucleosome free region encompassing the TSS (T. H. Kim et al., Nature 436, 876 (2005); M. G. Guenther, S. S. Levine, L. A. Boyer, R. Jaenisch, R. A. Young, Cell 130, 77 (2007); and A. Barski et al., Cell 129, 823 (2007). Comparison of available H3ac and H3K4me2 data in this cell line with GRO-seq suggested that both the upstream and downstream peaks of these histone modifications are associated with active transcription, with each peak of histone modifications being adjacent and downstream of an engaged polymerase (FIG. 25). Other studies have shown that histone modifications associated with transcription elongation (e.g. H3K36me3 and H3K79me3) do not associate in a bimodal fashion around TSSs (M. G. Guenther, S. S. Levine, L. A. Boyer, R. Jaenisch, R. A. Young, Cell 130, 77 (2007); A. Barski et al., Cell 129, 823 (2007). This and the lack of divergent GRO-seq reads further upstream indicated that the majority of divergent promoters experience initiation in the upstream direction, but that these polymerases do not productively elongate transcripts. Thus, promoters can distinguish polymerase in the forward versus the reverse direction.

To further assess the relationship between promoters identified by transcription factor binding (i.e. ChIP) assays and the presence of engaged polymerase, GRO-seq densities were compared with the list of over 10,000 active promoters identified in a previous study performed in the same cell line (T. H. Kim et al., Nature 436, 876 (2005). Active promoters in that study were identified genome-wide by binding of TAF1, a component of the general transcription factor TFIID that is critical for specifying most sites of initiation by Pol II (T. H. Kim et al., Nature 436, 876 (2005). That study identified 9,324 TFIID binding sites within 2.5 kb of annotated transcripts (referred to as transcript-matched) and 1,239 novel promoters that were greater than 2.5 kb from known 5′-ends of genes.

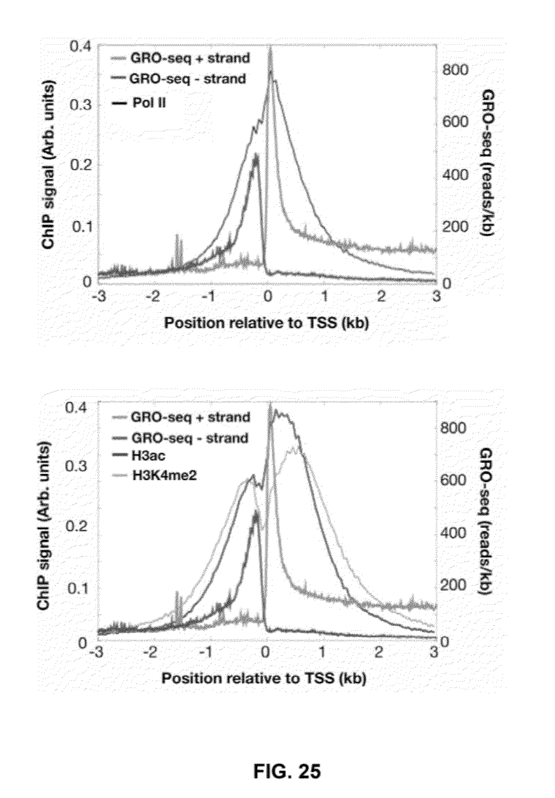

Of the promoters associated with annotated transcripts, 9,217 (98.9%) had coding-strand GRO-seq densities within the body of the associated gene significantly above background. Because the novel promoters had no associated orientation by ChIP, the neighboring +/−1 kb region was assayed. 1,185 (95.6%) had GRO-seq densities significantly above background. Details of the statistical methods are described in the Methods section below. GRO-seq not only confirmed these sites as active promoters, but also provided the direction and extent of transcription from these novel promoters (FIG. 26). When GRO-seq densities were used alone to identify the number of active promoters within +/−1 kb of RefSeq annotated 5′-ends, 16,882 active promoters were found. The increase in active promoters found here could be a consequence of different sensitivities, but may also reveal a class of promoters that are independent of TFIID (K. L. Huising a, B. F. Pugh, Mol. Cell 13, 573 (2004).

FIG. 26 shows an example of a novel transcription unit identified by GRO-seq. A novel transcription unit on chrX: 45,475,000-45,530,000 bp is shown that is not annotated by any of the major databases or gene prediction tools. The promoter was identified as putative by Pol II ChIP. GRO-seq confirms this as a promoter and identifies the direction of transcription.

The Kim et al. study also reported that Pol II was bound to 97% of confirmed TFIID binding sites by performing ChIP-chip with an antibody that recognizes Pol II (antibody: 8WG16). This represented the most comprehensive Pol II ChIP data set at the time of development of the present GRO-seq method. Thus, the IMR90 cell line was chosen.

The 8WG16 antibody preferentially recognized the hypophosphorylated form of the largest subunit of Pol II that was found at the 5′ ends of genes. It has been demonstrated at many genes that as Pol II progresses further into a gene it becomes hyperphosphorylated and thus a less suitable substrate for the antibody. Thus, in some cases the antibody will show a reduction in the downstream portions of a gene that actually reflects a reduced affinity for Pol II in these regions. Therefore, GRO-seq density and ChIP density cannot be directly compared in the downstream region of most genes, since GRO-seq detects transcriptionally engaged Pol H regardless of phosphorylation state. In addition, the array used to analyze the Pol II ChIP data was essentially a promoter array, so there is no data in the downstream portion of longer genes. The above reasons explain why, in some of the figures presented in Section 6.2 (Example 2) above and in this example, Pol II ChIP signal appeared concentrated only at the promoter regions, when this was a result of the antibody used and the extent of the array design.

6.2.3.9 Antisense Transcription in Gene Regions

A number of studies have reported that gene regions are transcribed in the reverse orientation with unanticipated high frequency. Transcript pairs have been identified that overlap at the 5′-ends, 3′-ends, or with full overlap (S. Katayama et al., Science 309, 1564 (2005); P. Kapranov, A. T. Willingham, T. R. Gingeras, Nat. Rev. Genet. 8, 413 (2007). Although antisense reads in gene regions accounted for only 6% of the total reads, ˜14,545 genes (58.7%) had antisense transcription significantly above background (P<0.01). Of these genes, 273 were accounted for by active annotated genes that overlapped at the 5′-end, 4,407 by active convergent genes with a maximum separation of 10 kb, and 242 by active annotated genes with full overlap (FIG. 15).

FIG. 15 shows a representative region that contains three types of antisense transcription (reverse direction from protein coding direction within genes) that are identified by GRO-seq. Three types of antisense transcription were identified by analysis of data generated by GRO-seq. A representative locus that shows three types of antisense transcription identified previously by others, and presently in this example. The number of occurrences of fully overlapping, 5′-overlapping, and 3′-overlapping (convergent) antisense transcription is shown below each.

6.2.4 Summary and Conclusions

This example presents the GRO-seq method for documenting transcribed regions in the human genome by isolation and large-scale sequencing of nascent RNAs. GRO-seq is efficient and requires only ˜5×10⁶ cells/library. The resulting NRO-cDNA library is highly enriched relative to total RNA. This technology can map polymerase locations with precision and allows the identification of active promoters and their directionality. The distribution of transcriptionally engaged polymerases around gene regions can identify interesting characteristics of promoters and gene regions such as promoter-proximal pausing, internal pausing, co-transcriptional cleavage of the nascent. RNA, the distance Pol II travels beyond annotated 3′ ends before termination, and the level antisense transcription within genes.

### 6.3 Example 3

### Identification of Transcription Start Sites

This example describes identification of transcription start sites using the GRO method. The 5′ ends of mRNAs are modified by addition of a 5-methyl guanosine cap to the 5′ phosphate. This modification is added naturally in vivo shortly after the initiation, and makes the 5′ end of the mRNA resistant to further modification by most enzymes. Capped NRO RNAs can be selected through an enzymatic enrichment by the oligo-capping method, a method well known in the art.

The NRO RNAs are first treated with calf intestinal alkaline phosphatase (CIAP), or other available phosphatases that suit this purpose, after the first round of BrU selection. This removes the 5′ phosphate from non-capped RNAs and effectively removes this class of RNA from the GRO-seq analysis because these molecules will no longer be substrates for the ligation reactions described above. Then, the CIAP is then inactivated and the capped RNAs are prepared for ligation by treatment with TAP. The remaining steps of the GRO-seq Method are then carried out as described above in Sections 6.2-6.3.

### 6.4 Example 4

### Identification of Polymerase Active Sites at Nucleotide Resolution

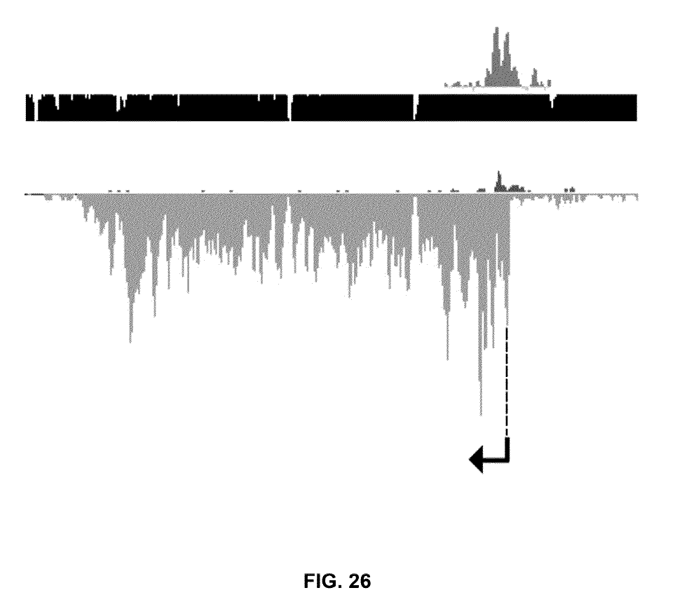

This example describes identification of polymerase active sites at nucleotide resolution using the GRO method. Isolated nuclei are subjected to RNase treatment prior to the step of performing the nuclear run-on reaction. Pol II protects 15-20 bases of nascent RNA upstream of the active site from RNase treatment, and is capable of resuming transcription when nucleotides are added. Analysis of RNase-pretreated run-ons using the GRO method described above locates the active site of the polymerase, which will be displaced 15-20 bases downstream of the observed 5′-end (FIG. 27).

FIG. 27 shows a schematic: for mapping the 3′-end of the engaged Pol II. Transcriptionally-engaged Pol II protects 15-20 by of the nascent transcripts, which could be further transcribed and to produce short run-on transcripts. Note that the 5′ end of the run-on transcript (marked as a star) maps the 3′ end of the transcript generated prior to the run-on analysis minus the 15-20 by Pol II protected site by RNase.

Alternatively, purifiable nucleotide analogs could be used that do not allow efficient elongation of polymerases after they are incorporated. In this case, when the polymerase incorporates the nucleotide analog, transcription will terminate one base downstream of where the active site was prior to the run-on. The terminated NRO-RNA can then be isolated, ligated with linkers, reverse transcribed, amplified, and then sequenced. Specific adapters can be used during the ligations that allow sequencing from either the 5′- or 3′-end. Due to the limitations of the length of reads of current sequencing technologies, sequencing from the 5′-end may not reach the end of the molecule, and thus not efficiently map the site of incorporation of the nucleotide analog. In this case, sequencing from the 3′-end is preferable since the first base sequenced will represent the site where the nucleotide analog was incorporated.

Nucleotide analogs that can be used for this embodiment include nucleotides with bulky moieties that will prevent the polymerase chain elongation due to interactions with the polymerase, which are well known in the art. Another analog well known in the art that can be used is a reversible terminating nucleotide analog. This analog lacks a 3′-OH group that is necessary for incorporating the next base. In this case, the 3′-end would be protected from polymerization to the next base. The protecting group is then be removed following isolation of the terminated NRO-RNA to allow the RNA to be ligated with a 3′ adapter.

The method of claim 1, wherein the purifiable nucleotide analog does not allow efficient elongation.

### 6.5 Example 5

### Mapping Sites of Cotranscriptional Cleavage Using GRO-seq

In another embodiment, the GRO-seq method can be used to map sites of co-transcriptional cleavage that delineate the 3′ end of mRNAs. Sites of co-transcriptional cleavage are detected by further adaptation of the methods described above. Prior to termination of transcription, the nascent RNA is cleaved by enzymes that recognize specific or desired sequences in the growing RNA chain. The cleavage site represents the 3′ end of the mRNA, and creates a new 5′ end that is associated with the transcribing polymerase. In order to detect this cleavage event, the NRO RNA is not base hydrolyzed, and the 5′ cap structure is not removed. In this case, only short, uncapped RNAs generated by polymerases that were less than 100 bases downstream of the cleavage site at the time of nuclei isolation are detected.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A method for performing a genome-wide nuclear run-on assay in a cell of interest comprising:
1) permeabilizing the cell of interest or isolating the nucleus from the cell of interest;
2) performing a nuclear run-on (NRO) reaction with the permeabilized cell or the isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration or duration of the NRO reaction;
4) isolating NRO-RNA from the NRO reaction;
5) hydrolyzing the NRO-RNA isolated from the NRO reaction to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA; and
8) ligating the hydrolyzed NRO-RNA to compatible adapter oligos.

2. The method of claim 1 wherein the cell of interest is a plurality of cells of interest and the step of permeabilizing comprises permeabilizing the plurality.

3. The method of claim 1 wherein the cell of interest is a plurality of cells of interest and the step of isolating the nucleus comprises isolating nuclei from the plurality.

4. The method of claim 1 wherein the step of isolating the nucleus comprises chemical or mechanical disruption of the outer cell membrane.

5. The method of claim 1 wherein the solid support is a bead support, column matrix, membrane support, biochip, microtiter plate or microfluidic device.

6. The method of claim 1 wherein the purifiable nucleotide analog comprises a purifiable affinity tag.

7. The method of claim 6 wherein the purifiable nucleotide analog is 5-Bromo-UTP (BrU) and the second nucleotide is not U or an analog thereof.

8. The method of claim 1 wherein the step of isolating the NRO-RNA comprises using a moiety that binds BrU contained within the NRO-RNA.

9. The method of claim 8 wherein the moiety is an antibody, an aptamer or a protein that reversibly binds BrU contained within the NRO-RNA.

10. The method of claim 1 wherein the step of enzymatically repairing the hydrolyzed NRO-RNA comprises removing the 5′ cap.

11. The method of claim 10 wherein removing the 5′ cap is accomplished through tobacco acid pyrophosphatase (TAP) treatment.

12. The method of claim 1 wherein the step of enzymatically repairing the hydrolyzed NRO-RNA comprises adding a 5′-phosphate (5′-P).

13. The method of claim 12 wherein adding the 5′-P is accomplished through neutral pH T4 polynucleotide kinase (T4 PNK) treatment.

14. The method of claim 1 wherein the step of enzymatically repairing the hydrolyzed NRO-RNA comprises removing a 3′-phosphate (3′-P).

15. The method of claim 14 wherein removing the 3′-P is accomplished through low pH T4 PNK treatment.

16. The method of claim 1 comprising reverse transcribing the NRO-RNA ligated to the compatible adapter oligos.

17. The method of claim 16 comprising producing a NRO-cDNA second strand by DNA extension.

18. The method of claim 17 comprising amplifying the double-stranded NRO-cDNA thereby producing a NRO-library.

19. The method of claim 18 comprising sequencing the amplified NRO-library.

20. The method of claim 19 comprising mapping one or more sequence reads to a reference genome.

21. The method of claim 20 comprising determining position, orientation or number of hits for the sequence read.

22. The method of claim 1 wherein the hydrolyzing step comprises base hydrolyzing.

23. The method of claim 1 wherein the hydrolyzing step comprises RNase hydrolyzing.

24. The method of claim 1 wherein the step of selecting hydrolyzed NRO-RNA comprises triple-selecting the hydrolyzed NRO-RNA.

25. The method of claim 1 comprising analyzing the hydrolyzed NRO-RNA ligated to compatible adapter oligos using sequencing analysis or microarray analysis.

26. The method of claim 25 wherein the sequencing analysis is massively parallel sequencing analysis.

27. The method of claim 25 wherein the analysis is microarray analysis and the NRO-RNA is ligated to an oligo containing a promoter for an RNA polymerase.

28. The method of claim 1 comprising analyzing production of nascent RNA.

29. The method of claim 28 comprising determining transcriptionally-engaged polymerase density.

30. The method of claim 28 wherein the production of nascent RNA is compared to accumulated mRNA levels to identify genes regulated by mRNA turnover.

31. A method for identifying a transcription start site in the genome of a cell of interest comprising the steps of:
1) permeabilizing the cell of interest or isolating the nucleus from the cell of interest;
2) performing a nuclear run-on (NRO) reaction with the permeabilized cell or the isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration or duration of the NRO reaction;
4) isolating NRO-RNA from the NRO reaction;
5) hydrolyzing the NRO-RNA isolated from the NRO reaction to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA;
8) selecting capped NRO-RNAs through enzymatic enrichment by the oligo-capping method; and
9) ligating the hydrolyzed NRO-RNA to compatible adapter oligos.

32. A method for identifying the position of an active site of an engaged RNA polymerase in the genome of a cell of interest comprising the steps of:
1) permeabilizing the cell of interest or isolating the nucleus from the cell of interest;
2) hydrolyzing RNA in the permeabilized cell or the isolated nucleus with an RNase;
3) performing a nuclear run-on (NRO) reaction with the permeabilized cell or the isolated nucleus, wherein a purifiable nucleotide analog is added to the NRO reaction;
4) optimizing the number of bases traveled by engaged polymerases for high resolution and low bias for nucleotide content of transcribed sequences by limiting a second nucleotide concentration or duration of the NRO reaction;
5) isolating NRO-RNA from the NRO reaction;
6) selecting hydrolyzed NRO-RNA with a solid support to obtain a highly enriched and purified fraction of the hydrolyzed NRO-RNA;
7) enzymatically repairing the hydrolyzed NRO-RNA by removing a 5′ cap from the NRO-RNA and adding a 5′-P to the NRO-RNA; and
8) ligating the hydrolyzed NRO-RNA to compatible adapter oligos.

33. The method of claim 32 wherein the step of enzymatically repairing the hydrolyzed NRO-RNA by removing the 5′ cap from the NRO-RNA and adding the 5′-P to the NRO-RNA comprises TAP treatment and neutral pH PINK treatment.

34. A method for mapping a site of co-transcriptional cleavage that delineates the 3′ end of an mRNA comprising the steps of:
1) permeabilizing the cell of interest or isolating the nucleus from the cell of interest;
2) performing a nuclear run-on (NRO) reaction with the permeabilized cell or isolated nucleus, wherein a purifiable nucleotide analog is added to the NRO reaction;
3) optimizing the number of bases traveled by engaged polymerases for high resolution and low bias for nucleotide content of transcribed sequences by limiting a second nucleotide concentration or duration of the NRO reaction;
4) isolating NRO-RNA from the NRO reaction;
5) optionally hydrolyzing the NRO-RNA isolated from the NRO reaction to optimize resolution of polymerase location;
6) selecting hydrolyzed NRO-RNA with a solid support to obtain a highly enriched and purified fraction of the hydrolyzed NRO-RNA;
7) enzymatically repairing the hydrolyzed NRO-RNA removing a 3′-P from the hydrolyzed NRO-RNA; and
8) ligating the hydrolyzed NRO-RNA to compatible adapter oligos.

35. The method of claim 1 comprising, after the step of ligating the hydrolyzed NRO-RNA to compatible adapter oligos, the step of amplifying the NRO-RNA.

36. The method of claim 35 comprising the step of:
performing reverse transcription after the step of ligating the hydrolyzed NRO-RNA to compatible adapter oligos;
wherein the ligating step comprises addition of a RNA oligomer to the 5′-end of the NRO-RNA and addition of an RNA oligomer to the 3′-end of the NRO-RNA.

37. The method of claim 1 comprising, after the step of amplifying the NRO-RNA, the step of purifying the amplified NRO-RNA by PAGE purification.

38. The method of claim 1 comprising:
treating the isolated nucleus with RNase prior to the step of running the NRO reaction; and
identifying polymerase active sites after the step of ligating the hydrolyzed NRO-RNA to compatible adapter oligos.

39. The method of claim 1, wherein the purifiable nucleotide analog does not allow further elongation.

40. The method of claim 32, wherein the purifiable nucleotide analog does not allow further elongation.

41. The method of claim 32 comprising analyzing production of nascent RNA.

42. The method of claim 41 comprising determining transcriptionally-engaged polymerase density.
